(19) United States
(12) Patent Application Publication
(10) Pub. No.: US 2024/0398869 A1
(43) Pub. Date: Dec. 5, 2024

(54) GENERATING ATRIAL AND VENTRICULAR CARDIOMYOCYTE LINEAGES FROM HUMAN PLURIPOTENT STEM CELLS

(71) Applicant: University Health Network, Toronto (CA)

(72) Inventors: Gordon Keller, Toronto (CA); Stephanie Protze, Toronto (CA); Jee Hoon Lee, Toronto (CA)

(73) Assignee: University Health Network, Toronto (CA)

(21) Appl. No.: 18/526,459

(22) Filed: Dec. 1, 2023

Related U.S. Application Data
(63) Continuation of application No. 18/186,807, filed on Mar. 20, 2023, now abandoned.
Application No. 18/186,807 is a continuation of application No. 17/957,895, filed on Sep. 30, 2022, now abandoned.
Application No. 17/957,895 is a continuation of application No. 16/466,278, filed on Jun. 3, 2019, now abandoned.
(60) Provisional application No. 62/430,815, filed on Dec. 6, 2016.
(60) Provisional application No. 62/429,823, filed on Dec. 4, 2016.

(51) Int. Cl. A61K 35/28; A61K 35/34; A61P 9/04; C12N 5/077; G01N 33/50; G01N 33/68

(52) U.S. Cl. CPC A61K 35/28; A61K 35/34; A61P 9/04; C12N 5/0657; G01N 33/5014; G01N 33/5061; G01N 33/6887

(57) ABSTRACT

Methods are disclosed for producing populations of cardiomyocytes from pluripotent stem cells. Populations may be enriched for either atrial or ventricular cardiomyocytes and the resulting ventricular population may be essentially free of pacemaker cells. The method includes incubating pluripotent stem cells in a suitable medium with a BMP component, and an activin component, the amounts of activin may be varied to enrich for either atrial or ventricular cardiomyocytes. The enriched populations, as well as methods of using the same to treat patients in need of cardiac repair are disclosed.

### CROSS REFERENCE TO RELATED APPLICATIONS

This application claims priority under 35 U.S.C. § 119 to U.S. Provisional Application Ser. No. 62/429,823 filed Dec. 4, 2016 and to U.S. Provisional Application Ser. No. 62/430,815 field Dec. 6, 2016. The entire contents of these earlier-filed patent applications are hereby expressly incorporated herein by reference including, without limitation, each of the specification, claims, and abstract, as well as any figures, tables, or drawings thereof.

### FIELD

The disclosure provides methods for producing and compositions comprising enriched populations of atrial cardiomyocytes, ventricular cardiomyocytes, and use of same for therapeutic treatment, disease modeling, drug discover, as well as biomarkers and methods for identifying these enriched subpopulations.

### BACKGROUND

The goals of heart disease research are to understand in greater detail what happens in heart disease and why, and to find ways to prevent damage or to repair or replace damaged heart tissue. Existing therapies are aimed at slowing progression of heart failure rather than restoring lost contractile function. At present, the only available therapeutic option to replace the lost contractile function is whole organ transplantation, but because demand greatly exceeds supply, there has been considerable Interest in stem cell-based therapies as an alternative approach. Of particular use would be the ability to utilize cardiomyocytes differentiated from stem cells for purposes of transplantation. Various studies have demonstrated that use of human embryonic stem cell (hESC) derived cardiomyocytes, once transplanted, can remuscularize injured hearts and mediate improvements in contractile function (see for example Shiba et al. (2012). One of the challenges however has been the mixed nature of the stemcell-derived cardiomyocyte populations, which may be responsible for problems such as e.g. graft-related ventricular tachyarrhythmias. What is needed is the ability to further differentiate stem cells to allow for the formation of enriched populations of particular subtypes of cardiomyocytes, such as ventricular cardiomyocytes and atrial cardiomyocytes and to allow these enriched populations of cardiomyocytes to be used for purposes of treatment.

### SUMMARY

In an aspect, there is provided a method of producing a population of cardiomyocytes enriched for atrial cardiomyocytes, the steps comprising: i. incubating pluripotent stem cells in a mesoderm induction medium said mesoderm induction medium comprising at least a BMP component, optionally BMP4, and an effective amount of an activin component, optionally Activin A, to generate atrial mesoderm. In this aspect, the method comprises further adding a retinoic add component to the cells, said addition of retinoic acid added during the mesoderm induction or cardiovascular specification stage, and culturing said cells so that a population of cardiomyocytes enriched for atrial cardiomyocytes is generated.

In one aspect that atrial mesoderm may be characterized by said cells being one or more of RALDH2 positive CD235 negative, and CYP26A1 negative In an embodiment the BMP component to the activin component is provided in a ratio of 3:2. In another embodiment the activin component is present in an amount of about 0.001 ng/ml to 6 ng/ml and said BMP component is present in an amount of from about 3 ng/ml to about 100 ng/ml.

In an aspect, there is provided a method of producing a population of cardiomyocytes enriched for ventricular cardiomyocytes, the steps comprising: incubating the pluripotent stem cells in a mesoderm induction medium comprising a BMP component, optionally BMP4, and an effecive amount of an activin component, optionally Activin A, sufficient to generate ventricular mesoderm and thereafter, culturing said cells in a medium(s) suitable to generate a population of cardiomyocytes enriched for ventricular cardiomyocytes. In an embodiment the amount of activin component effective to generate ventricular mesoderm is characterized by said ventricular mesoderm being one or more of RALDH2 negative, CD235a positive, and CYP26A1 positive. In another embodiment the concentration of the activin component is greater than the concentration of the BMP component. In an embodiment activin component is present in an amount of about 6 ng/ml to 20 ng/ml and said BMP is present in an amount of from about 3 ng/ml to about 20 ng/ml.

In an aspect, there is provided a population of cardiomyocytes enriched for ventricular cardiomyocytes, wherein said population is essentially free of pacemaker cells. In another aspect the population is devoid of pacemaker cells. In another aspect there is provided an isolated population of cardiomyocytes: enriched for ventricular cardiomyocytes comprising at least or about 50% of ventricular cardiomyocytes, at least or about 60% of ventricular cardiomyocytes, at least or about 70% of ventricular cardiomyocytes, at least or about 80% of ventricular cardiomyocytes, at least or about 90% of ventricular cardiomyocytes, at least about 95% of ventricular cardiomyocytes, or at least about 99% ventricular cardiomyocytes, preferably obtained according to the method described herein. In an aspect of the invention, the isolated population is essentially free or pacemaker cells (less than 5% of total cells). In a preferred embodiment the population includes less than 1% pacemaker cells, less than 0.5% pacemaker cells, less than 0.1% pacemaker cells, less than 0.01% pacemaker cells, less than 0.001% pacemaker cells, 0.0001% pacemaker cells, or is completely devoid of pacemaker cells. While not wishing to be bound by any theory it is postulated that the presence of pacemaker cells may induce independent and separate contraction of muscle when introduced to a patient In a preferred embodiment, pacemaker cells are not detectable in the isolated population of ventricular cardiomyocytes using currently available techniques.

In an aspect, there is provided an isolated population of cardiomyocytes enriched for atrial cardiomyocytes comprising at least or about 50% of atrial cardiomyocytes, at least or about 60% of atrial cardiomyocytes, at least or about 70% of atrial cardiomyocytes, at least or about 80% of atrial cardiomyocytes, or at least or about 90% of atrial cardiomyocytes, or at least or about 95% atrial cardiomyocytes, or at least or about 99 atrial cardiomyocytes, preferably obtained according to the method described herein.

In an aspect, there is provided a method of treating a subject in need of cardiac repair, such as, for example, a subject with heart failure, or a subject at risk of heart failure, comprising administering to the subject the population of ventricular cardiomyocytes described herein.

In an aspect, there is provided the population of ventricular cardiomyocytes described herein, for use in the treatment of a subject in need of cardiac repair, such as, for example, a subject with heart failure or a subject at risk of heart failure.

In an aspect, there is provided use of the population of ventricular cardiomyocytes described herein, in the preparation of a medicament for the treatment of a subject in need of cardiac repair, such as, for example, a subject with heart failure or a subject at risk of heart failure.

In an aspect, there is provided a process for detecting atrial mesoderm in a population of cells, comprising detecting ALDH, preferably RALDH2, wherein a presence of ALDH, preferably RALDH2, is indicative of atrial mesoderm.

In an aspect, there is provided a process for detecting ventricular mesoderm in a population of cells, comprising detecting one or more of CD235a, CD235b, and CYP26A1, wherein a presence of CD235a, CD235b, and/or CYP26A1 is indicative of ventricular mesoderm.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings.

FIG. 1. RA signaling Promotes Atrial-like Cardiomyocyte Development. (A) Schematic of the hPSC cardiomyocyte differentiation protocol indicating developmental stages and timing of RA addition. (B and C) qRT-PCR analysis of the expression levels of (B) a pan-cardiomyocyte gene and (C) ventricular-specific (MYL2), and atrial-specific (KCNJ3) genes in NKX2-5⁺SIRPa⁺CD90⁻ cells isolated from day 20 EB populations induced with 10 ng/mL BMP4 and 6 ng/mL Activin A (10B/6A) and treated with RA at the indicated time points (n=3) and in fetal tissue controls (n=6) (t test, *p<0.05 and **p<0.01 versus DMSO control and ^{##}p<0.01 F-V versus F-A). (D) Heatmap comparing the gene expression profiles of NKX2-5⁺SIRPa⁺CD90⁻ cells isolated from day 20 EBs (10B8A induced) and treated with RA or DMSO (control) between days 3 and 5 (n=5). Values represent log₁₀ of expression levels relative to the housekeeping gene TBP. (E) Representative low cytometric analyses of the proportion of NKX2-5⁺/CTNT⁺ and MLC2V⁺/CTNT⁺ cells in day 20 EB populations induced with 10B/6A and treated between days 3 and 5 with RA or DMSO (control). (F) Bar graph showing the average proportion of MLC2V⁺CTNT⁺ cells In day 20 EBs treated as indicated (t test, **p<0.01 versus DMSO control; n=4). (G and H) Photomicrograph showing immunostaining of (G) MLC2V and (H) COUPTFII in day 20 EBs (10B/6A induced) treated with either DMSO (control) or RA between days 3 and 5. Cells were co-stained with CTNT to identify all cardiomyocytes and DAPI to visualize all cells. Scale bars represent 100 mm. For all PCR analyses, expression values were normalized to the housekeeping gene TBP. Error bars represent SEM. F-V, fetal ventricular tissue; F-A, fetal atrial tissue. See also FIG. 8.

FIG. 2. Induction of ALDH⁺ Cardiogenic Mesoderm (A) Representative flow cytometric analyses of ALDH activity in PDGFRalpha+ mesoderm on 10B/6A-induced EBs. ALDH inhibitor (DEAB)-treated cells were used as a control. (B and C) Representative flow cytometric analyses of day 4 ALDH activity and PDGRalpha expression (left columns) and corresponding day 20 CTNT expression following manipulation (days 1-3) of (B) Activin A concentrations (0,110 ng/mL in the presence of 10 ng/mL BMP4 or (C) BMP4 concentrations (1-10 ng/mL in the presence of 2 ng/mL Activin A. (D) Representative flow cytometric analyses of ALDH activity and PDGFRalpha expression in EBs induced with 3B/2A. (E)qRT-PCR analyses of the expression levels of ALDH1A2 and CYP26A1 in 10B/6A- and 3B/2A-induced EB populations (t test, *p<0.05 and **p<0.01 versus 10B/6A-induced EBs at corresponding differentiation days; n=4). For all PCR analyses, expression values were normalized to the housekeeping gene TBP. Error bars represent SEM. See also FIG. 9.

FIG. 3. Retinol specifies AF+ mesoderm to an Atrial Fate (A) Schematic of the strategy used for the isolation and analyses of the cardiogenic potential of the ALDH⁺ PDGFRa⁺(fraction I) and ALDH⁻PDGFRa⁺ (fraction II) fractions isolated from day 4 EBs induced with 3B/2A. (B) Representative flow cytometric plot showing the cell-sorting strategy used to isolate the ALDH+ PDGFRa+ (fraction I) and ALDH−PDGFRa+ (fraction II) fractions. (C) qRT-PCR analyses of ALDH1A2 expression within the isolated populations indicated above (t test, **p<0.01; n=3). (D and E) Flow cytometric analyses of the proportion of (D) CTNT+ and (E) MLC2V+ cells in day 20 populations generated from ROH-, RA-, or DMSO (control)-treated day 4 ALDH+ PDGFRa+ and ALDH− PDGFRa+ fractions (t test, *p<0.05 and *p<0.01 versus DMSO control; n=6). (F and G) qRT-PCR analysis of the expression levels of (F) ventricular and (G) atrial genes in the day 20 populations of indicated treatment groups (n=6) (t test, *p<0.05 and **p<0.01 versus DMSO control). For all PCR analyses, expression values were normalized to the housekeeping gene TBP. Error bars represent SEM. WNTi, WNT inhibition; ROH, retinol. See also FIG. 10.

FIG. 4. CD235a Expression Marks Mesoderm with Ventricular Potential (A) Representative flow cytometric analyses of CD235a expression and ALDH activity in EBs Induced with either 10B/6A (top) or 3B/2A (bottom). (B) Representative flow cytometric plot showing the cell-sorting strategy used for isolating the CD235a⁺ (fraction Ill, ventricular potential) and ALDH⁺ (fraction IV, atrial potential) fractions from 5B/4A-induced EBs at day 4. (C and D) Flow cytometric analyses of the proportion of (C) CTNT⁺ and (D) MLC2V+ cells in day 20 populations generated from the day 4 ALDH⁺ and CD235a fractions treated for 24 hr with ROH, RA, or DMSO (control) (t test, *p<0.05 and **p<0.01 versus DMSO control and ^{##}p<0.01 versus indicated sample; n=5). (E and F) qRT-PCR analyses of the expression levels of (E) ventricular and (F) atrial genes in day 20 populations generated from the day 4 ALDH⁺ and CD235a⁺ fractions treated as indicated (n=5) (t test, *p<0.05 and **p<0.01 versus DMSO control, ^{#}p<0.05 and ^{##}p<0.01 versus indicated sample). For al PCR analyses, expression values were normalized to the housekeeping gene TBP. Error bars represent SEM. See also FIG. 11

FIG. 5 Optimization of CD235a⁺ Cardiogenic Mesoderm Induction (A and B) Representative flow cytometric analyses of day 4 ALDH activity and CD235a expression (left columns) and corresponding day 20 MLC2V and CTNT expression (right columns) following the manipulation (days 1-3) of (A) Activin A concentrations (2-20 ng/mL) in the presence of 10 ng/mL BMP4 or (B) BMP4 concentrations (3-20 ng/mL) in the presence of 12 ng/mL Activin A. (C) Representative flow cytometric plots showing the proportion of ALDH activity and CD235a expression in day 4 5B/12A-(top) and 3B/2 A-induced EBs (bottom). (D and E) Flow cytometric analyses of the proportion of (D) CTNT+ and (E) MLC2V+ cells in day 20 EB populations from 5B/12A- or 3B/2A-induced EBs treated with ROH, RA, or DMSO (control) for 48 hr (days 3-5) (t test, *p<0.05 and **p<0.01 versus DMSO control; n=4). (F and G) qRT-PCR analyses of the expression levels of (F) ventricular and (G) atrial genes in day 20 EB populations generated with the Indicated treatments (n=4) (t test, *p<0.05 and **p<0.01 versus DMSO control). (H) Representative flow cytometric analyses of the proportion of NKX2-5-CTNT+ cells in day 20 EB populations induced with 5B/12A or 3B/2A. (I) Quantification of spontaneous beating rates of day 20 EBs induced with 5B/12A or 3B/2A (n=17) (t test, **p<0.01). (J) Bar graph showing the average proportion of NKX2-5-CTNT+ cells in day 20 EB populations induced with 5B/12A, 10B/6A, or 3B/2A (days 1-3) in the presence or absence of RA (0.5 mM, days 3-5) (t test, *p<0.05 versus indicated sample; n=5). For all PCR analyses, expression values were normalized to the house-keeping gene TBP. Error bars represent SEM. See also FIG. 12

FIG. 6 Comparison of Cardiomyocytes Derived from Different Mesoderm Populations (A and B) qRT-PCR analysis of the expression levels of (A) pan-cardiomyocyte and (B) ventricular genes in NKX2-5+SIRPa+CD90− cells isolated from day 20 EBs Induced under ventricular Induction (VI). mixed induction (MI also referred to as MM), and atrial induction (AI) conditions (n=5) and in fetal tissue controls (n=6) (t test, *p<0.05 and **p<0.01 versus indicated sample, ##p<0.01 F-V versus F-A). (C) qRT-PCR analyses of the expression levels of atrial genes in NKX2-5⁺SIRPa⁺CD90⁻ cells Isolated from day 20 non-treated or RA-treated EBs (days 3-5) induced as Indicated (n=4) (t test, *p<0.05 and **p<0.01 VI versus VI+RA. (D) Photomicrograph showing immunostaining of COUPTFII in NKX2-5⁺SIRPa⁺CD90⁻ cells isolated from day 20 EBs induced with VI+RA or AI+RA. Cells were co-stained with CTNT to identify al cardiomyocytes and with DAPI to visualize all cells. Scale bars represent 100 mm. (EG) AP measurements in NKX2-5SIRPa⁺CD90⁻ cardiomyocytes isolated from day 20 EBs induced as indicated. (E) Representative recordings of spontaneous APs in individual cardiomyocytes isolated from the indicated groups. (F) Quantification of AP duration at 30%/90% repolarization (APD30/90) in cardiomyocytes isolated from VI (n=18), VI+RA (n=18), and AI+RA (n=20) EBs (t test, *p<0.05 and **p<0.01 versus indicated sample). (G) Bar graph showing the proportion of atrial (APD30/90<0.3), ventricular (APD30/90 R 0.3), and immature (maximal upstroke velocity [dv/dtₘₐₓ]<10 and cycle length [CL] R 1) cardiomyocytes in each group based on analyses of recorded APs. (H-J) Analysis of acetylcholine-activated inward rectifier potassium current densities (I_{KACh}) in cardiomyocytes isolated from EBs induced as indicated. (H) Representative recording showing the carbachol (CCh)-sensitive current (I_{KACh}) in a cardiomyocyte isolated from AI+RA-induced EBs, quantified as the difference between the current measured after (CCh) and before (control) application of 10 mM CCh (inset voltage protocol). (I) Current-voltage relationship for I_{KACh} current densities in ventricular cardiomyocytes (validated ventricular-like AP shape) isolated from VI EBs and in atrial cardiomyocytes (validated atrial-like AP shape) isolated from VI+RA and AI+RA EBs. (J) Quantification of maximum I_{KACh} current densities recorded at −120 mV in each group (t test, *p<0.05 and **p<0.01 versus indicated sample). For all PCR analyses, expression values were normalized to the housekeeping gene TBP. Error bars represent SEM. F-V, fetal ventricular tissue; F-A, fetal atrial tissue; n.s., not significant. See also FIG. 13.

FIG. 7. Generation of Ventricular and Atrial Cardiomyocytes from Other hPSC Lines (A) Representative flow cytometric analyses of ALDH activity and CD235a expression in day 4 HES2-derived EBs induced under ventricular (5B/6A, top) or atrial (5B/2A, bottom) conditions. (B) Representative flow cytometric analyses of CTNT and MLC2V expression in corresponding day 20 EB populations generated under ventricular or atrial conditions and subjected to ROH, RA, or DMSO (control) treatment from days 3 to 5. (C and D) qRT-PCR analyses of the expression levels of (C) ventricular and (D) atrial genes in SIRPa⁺CD90⁻ cells isolated from day 20 EBs induced under the indicated conditions (t test, *p<0.05 versus DMSO control, ^{#}p<0.05 and ^{##}p<0.01 versus indicated sample; n=5). (E) Representative flow cytometric analyses of ALDH activity and CD235a expression in day 4 MSC-iPS1-derived EBs induced under ventricular (48/4A, top) or atrial (4B/1A+SB, bottom) conditions. (F) Representative flow cytometric analyses of CTNT and MLC2V expression in corresponding day 20 EB populations generated in ventricular or atrial conditions and subjected to ROH, RA, or DMSO (control) treatment from days 3 to 5. (G and H) qRT-PCR analyses of the expression levels of (G) ventricular and (H) atrial genes in SIRPa+CD90− cells isolated from day 20 EBs induced as indicated (t test, *p<0.05 and **p<0.01 versus DMSO control, ##p<0.01 versus Indicated sample; n=5). (I) Model of human atrial and ventricular cardiomyocyte development from hPSCs. In this model, distinct mesoderm populations defined by CD235a and CYP26A1 expression or RALDH2 expression and ALDH activity are induced by different concentrations of Activin A and BMP4. The RALDH2+ALDH+, but not the CD235a+CYP26A1+, mesoderm can respond to ROH to generate atrial-like cardiomyocytes. RA can specify both mesoderm populations to an atrial fate. However, specification from the CD235a+ mesoderm is less efficient than from the RALDH2+ mesoderm and the resulting atrial phenotype is suboptimal. In the absence of retinoid signaling (ROH, RA), the RALDH2+ mesoderm can give rise to ventricular cardiomyocytes with low efficiency. For all PCR analyses, expression values were normalized to the housekeeping gene TBP. Error bars represent SEM. SB, SB-431542 (Nodal/Activin A/TGF-b inhibitor); WNTi, WNT inhibition. See also FIG. 14.

FIG. 8. Related to FIG. 1. Generation of atrial-like cardiomyocytes from hPSCs. (A) Representative flow cytometric plot showing the cell sorting strategy used for the isolation of SIRPalpha⁺ NKX2-5⁺CD90⁻ cardiomyocytes at day 20 of differentiation. (B-E) Graphs of the QRT-PCR analysis represented as a heat map in FIG. 1D showing the expression levels of (B) pan-cardiomyocyte, (C) ventricular cardiomyocyte, (D) atrial cardiomyocyte, (E) cardiac ion channel and connexin channel genes in SIRPalpha⁺NKX2-5⁺CD90⁻ cardiomyocytes isolated from EBs induced with 10B/6A and treated with either RA or DMSO (control) between days 3 and 5 of differentiation (n=5). t-test: *P<0.05, **P<0.01 vs. DMSO-control, ^{##}P<0.01 F-V vs. F-A. (F) QRT-PCR analyses of the expression levels of retinoic acid receptor isoforms (RARA, RARB, and RARG) in whole EB populations induced with 10B/6A at the indicated days of differentiation (n=3). (G) Flow cytometric analyses of the proportion of CTNT⁺ cells in day 20 EBs treated between days 3-5 with either DMSO (Control), RA or the receptor-specific agonists (n=3). (H) QRT-PCR analyses of the expression levels of ventricular-specific gene MYL2 in day 20 EBs treated between days 3 and 5 with the indicated treatments (n=3). t-test: **P<0.01 vs. DMSO-control. For al PCR analyses, expression values were normalized to housekeeping gene TBP. Error bars represent SEM. F-V: fetal ventricular tissue, F-A fetal atrial tissue, RA-retinoic acid, AM580: RARalpha-agonist, AC55649: RAR-agonist, CD437: RARγ-agonist.

FIG. 9. Related to FIG. 2. Developmental kinetics of 10B/6A- and 3B/2Ainduced mesoderm. (A) Bar graph showing the average number of cells generated per well of a 6-well plate of EBs (day 20) induced with 31/2A and 10B/6A (n=5). (B) QRT-PCR analyses of the expression kinetics of ALDH1A1 and ALDH1A3 aldehyde dehydrogenase isoforms in EBs at the Indicated days of differentiation following the induction (days 1-3) with either 3B/2A or 10B/6A (n=3). (C) QRT-PCR analyses of the expression kinetics of the primitive streak marker T (Bachyury) and cardiogenic mesoderm marker MESP1 In EBs at the Indicated days of differentiation following the Induction (days 1-3) with either 3B/2A or 10B/6A (n=4). For all PCR analyses, expression values were nornalized to housekeeping gene TBP. Error bars represent SEM.

FIG. 10. ALDH activity in 3B/2A-induced mesoderm populations. Representative flow cytometric analyses of ALDH activity following 24 hour culture as aggregates of ALDH⁺ PDGFRalpha⁺ (fraction I) and ALDH⁻ PDGFRalpha⁺ (fraction II) calls isolated from day 4 EBs induced with 3B/2A.

FIG. 11. Analysis of GYPA expression in unsorted and sorted mesoderm populations. (A)QRT-PCR analyses of the expression levels of GYPA in 3B/2A and 10B/6A-induced EBs at the Indicated days of differentiation. t-test: **P<0.01 vs. indicated sample (n=4). (B) QRT-PCR analyses of the expression levels of ALDH1A2, CYP26A1, and GYPA in ALDH⁺ (fraction IV) and CD235a⁺(fractionIII) fractions isolated from day 4 EBs induced with 5B/4A. For all PCR analyses, expression values were normalized to housekeeping gene TBP. t-test: **P<0.01 (n=3). Error bars represent SEM.

FIG. 12. Optimization of ventricular differentiation through manipulation of mesoderm induction.

(A and B) Flow cytometric analyses of the proportion of CD235a⁺ cells in day 4 EBs (left) and resulting CTNT⁺MLC2V⁺ cells in day 20 EBs (right) following the manipulation (days 1-3) of: (A) Activin A concentrations (2-20 ng/ml) in the presence of 10 ng/ml BMP4 or (B) BMP4 concentrations (3-20 ng/ml) in the presence of 12 ng/ml Activin A (n=6). t-test *P<0.05, **P<0.01 vs. indicated sample. (C) Bar graph showing the average number of cells generated per well of a 6-well plate of EBs (day 20) induced with either 5B/12A or 10B/6A (n=4). t-test: P>0.05=n.s., not significant. (D) Flow cytometric analyses of the proportion of CTNT⁺MLC2V⁺ cells at day 20 and 40 of culture in EB populations induced as indicated (n=3). t-test: P>0.05=n.s., not significant Error bars represent SEM.

FIG. 13. Characterization of atrial and ventricular cardiomyocytes derived from different mesoderm populations. (A) Flow cytometric analysis of the proportion of MLC2V⁺ cells in day 20 EBs induced under ventricular Induction (VI), mixed induction (MI) and atrial induction (AI) conditions. t-test: **P<0.01 vs. indicated sample. (B) Photomicrograph showing immunostaining of MLC2V in day 20 EB populations generated from AI and VI. Cells were co-stained with CTNT to Identify all cardiomyocytes and DAPI to visualize all cells. Scale bars represent 100 μm. (C and D) QRT-PCR analyses of the expression levels of (C) atrial and (D) pacemaker genes In NKX2− 5⁺SIRPalpha⁺CD90⁻ cells Isolated from day 20 EB populations induced as Indicated (n=4) and In fetal tissue controls (n=8). t-test: *P<0.05, **P<0.01 VI vs. VI+RA; AI vs. AI+RA and vs. Indicated samples. ^{##}P<0.01 F-V vs. F-A. (E) Flow cytometric analyses of the proportion of NKX2-5⁺SIRPalpha⁺ cells in day 20 EBs Induced under either VI or AI conditions and treated with the Indicated concentrations of RA (0.125-4 μM) between days 3 and 5 (n=3). (F-H) QRT-PCR analyses of the expression levels of (F) the atrial gene KCN5A (G) the ventricular genes MYL2, IRX4 and (H) the atrial genes KCNJ3, CACNA1D and NR2F2 in NKX2-5⁺SIRPalpha⁺CD90⁻ cells isolated from the different day 20 populations. t-test: *P<0.05, **P<0.01 vs. VI sample at the respective RA concentration (n=4). For all PCR analyses, expression values were normalized to housekeeping gene TBP. Error bars represent SEM. F-V: fetal ventricular tissue, F-A: fetal atrial tissue, n.s.: not significant.

FIG. 14 Characterization of atrial and ventricular cardiomyocytes derived from HES2 and MSC-iPS1 hPSCs. (A)Representative flow cytometric analysis of ALDH activity and CD235a expression in MSC-iPS1-derived EBs induced with 4B/1A and subsequently treated with or without SB-431542 (SB) (days 3-5). (B-D) QRT-PCR analyses of the expression levels of (B) pan-cardiomyocyte, (C) ventricular and (D) atrial genes in the SIRPalpha⁺CD90⁻ cells isolated from day 20 HES2-derived EB populations induced under ventricular (5B/6A) or atrial (5B/2A) conditions (days 1-3) and treated between days 3 and 5 with either ROH, RA or DMSO (Control). t-test: *P<0.05, **P<0.01 vs. DMSO-control, ^{#}P<0.05, ^{##}P<0.01 vs. Indicated sample (n=5). (E-G) QRT-PCR analyses of the expression levels of (E) pan-cardiomyocyte, (F) ventricular and (G) atrial genes in SIRPalpha⁺CD90⁻ cells isolated from day 20 MSC-iPS1-derived EBs induced under ventricular (4B/4A) or atrial (4B/1A+SB) conditions (days 1-3) and treated between days 3 and 5 with either ROH, RA or DMSO (Control). t-test *P<0.05, ^{#}P<0.01 vs. DMSO-control, ^{##}P<0.01 vs. indicated sample (n=5). For al PCR analyses, expression values were normalized to housekeeping gene TBP. Error bars represent SEM. SB: SB-431542 (Nodal/Activin A/TGFβ inhibitor)

FIG. 15. A schematic depicting various differentiation pathways for cardiac cells.

### DETAILED DESCRIPTION

### Definitions

The term “ventricular cardiomyocytes” as used herein refers to a population of cells enriched for ventricular cells, or enriched for cells which have ventriculocyte properties. These include cardiomyocytes expressing ventricular specific markers such as MYL2, IRX4, and/or elevated levels of NKX2-5 and/or display electrophysical properties of a ventricular cell (e.g. action potential).

The term “atrial cardiomyocytes” as used herein refers to a population of cells enriched for atrial cells or enriched for cells which have atrial cell like properties. These include cardiomyocytes expressing atrial specific markers such as the atrial ion channel gene KCNJ3, NPPA, GJA5 and/or MYL7 and/or display electrophysical properties of an atrial cell (e.g. action potential).

The terms “cardiovascular mesoderm cells” and “cardiovascular mesoderm” as used herein refer to a population of mesoderm cells enriched for mesoderm cells having increased potential for differentiation into cardiovascular cells relative to other mesoderm cells.

The terms “ventricular mesoderm cells” and “ventricular mesoderm” as used herein refer to a population comprising mesoderm cells enriched for mesoderm cells having increased potential for differentiation into ventricular cardiomyocytes relative to other mesoderm cells. These include mesoderm cells that are one or more of ALDH−, RALDH2− CD235a+, CD235b+, and CYP26A1+.

The terms “atrial mesoderm cells” and “atrial mesoderm” as used herein refer to a population comprising mesoderm cells enriched for mesoderm cells having increased potential for differentiation into atrial cardiomyocytes relative to other mesoderm cells. These include mesoderm cells that are one or more of ALDH+, RALDH2+, CD235a−, CD235b−, and CYP26A1−.

The term “cardiomyocyte” as used herein is a cardiac lineage cell. Cardiac lineage cells typically express the pan cardiac specific marker cTNT.

The term “pacemaker cell” as used herein refers to a cardiomyocyte, which has pacemaker activity and expresses sinoatrial nodal (SAN) cell specific markers. Pacemaker cells generally have faster beating rates than ventricular cardiomyocytes. Pacemaker cells do not express NKX2-5.

The term “NKX2-5” as used herein refers to the cardiac homeobox protein NKX2-5 encoded in humans by the NKX2-5 gene. The gene is involved in cardiac differentiation and is expressed in cardiomyocyte subtypes such as ventricular cardiomyocytes. Expression of NKX2-5 can be measured using for example an antibody specific to NKX2-5 or for example by using a NKX2-5 reporter construct.

The term “BMP component” as used herein means any molecule, optionally any BMP or growth and differentiation factor (GDF), or small molecule, that activates the receptor for BMP4, including for example BMP4 and/or BMP2.

The term “BMP4” (for example Gene ID: 652) as used herein refers to Bone Morphogenetic Protein 4, for example human BMP4, as well as active conjugates and/or fragments thereof, that can for example activate BMP4 receptor signaling.

The term “essentially free of pacemaker cells” as used herein refers to ted a population of cardiomyocytes wherein pacemaker cells comprise less than 5% of total cells, less than 1% pacemaker cells, less than 0.5% pacemaker cells, less than 0.1% pacemaker cells, less than 0.01% pacemaker cells, less than 0.001% pacemaker cells, or less than 0.0001% pacemaker cells, is completely devoid of pacemaker cells, or wherein pacemaker cells are not detectable in the population of cardiomyocytes via currently available methods of detection. While not wishing to be bound by any theory it is postulated that the presence of pacemaker cells in a population of ventricular cells may induce independent and separate contraction of muscle when introduced to a patient.

The term “activin component” as used herein means one or more components, or a composition comprising said component(s), that activates nodal signal transduction, optionally which has Activin A activity such as Activin A and/or nodal.

The term “activin” or “ActA” as used herein refers to “Activin A”, (e.g. Gene ID: 3624), for example human Activin A, as well as active conjugates and fragments thereof or small molecules, that can activate nodal signal transduction.

The term “retinoic acid” or “RA” signifies retinoic acid.

The term “retinoic acid component” includes compounds that mediate the function of vitamin A, and includes for example all-trans RA (e.g. Sigma R2625), 9-cis RA (e.g. Sigma R4643), and retinol (e.g. Sigma R7632) as well as RA analogs (e.g. RAR agonists), such as AM580, a selective RARalpha agonist (Tocns 0760), AC55549, a selective RARP agonist (Tocris 2436), and CD437, a selective RARγ agonist (Tocris 1549)

The term “embryoid body medium” as used herein is a culture medium that supports formation of aggregates (e.g. floating aggregates of PSCs having the potential to differentiate into cells of all three germ layers) or embryoid bodies of PSCs, and comprises a minimal media such as StemPro 34 (ThermoFisher), MesoFate™ (Stemgent), RPMI (ThermoFisher and other companies), HES-media (DMEM/F12 with KnockOut Serum Replacement, ThermoFisher and other companies) and for example a BMP component, optionally BMP4, and further optionally comprising a Rho-associated protein kinase (ROCK) inhibitor.

The term “embryoid body aggregation phase” as used herein means the time period non-aggregated hPSCs are cultured for example with an embryoid body medium described herein and are treated with BMP component and as well as optionally ROCK inhibitor and/or other components that result in aggregates, such as embryoid bodies (e.g., aggregates of PSCs that can be differentiated into cells of all three germ layers). The component treatments can be simultaneous, overlapping or distinct. For example, a first component can be comprised in the medium and a second component can be added to the medium during the embryoid body aggregation phase.

The term “mesoderm induction medium” can include a culture medium that supports the formation of cardiovascular mesoderm cells and comprises a minimal media such as StemPro 34 (ThermoFisher), MesoFate™ (Stemgent), RPMI (ThermoFisher and other companies). Mesoderm induction medium can include additional components such as a BMP component, optionally BMP4, an activin component, optionally Activin A, and may include other components such as bFGF. Depending upon the desired fate of the cardiomyocyte cells produced from the mesoderm, different concentrations of each of the BMP component and activin component may be adjusted as taught herein.

The term “mesoderm induction phase” can describe the time period in which PSCs are cultured with mesoderm induction medium, including treatment with BMP component and an activin component as well as optionally an FGF component and/or other components, such that PSCs differentiate into mesoderm cells. The BMP and activin component treatments can be simultaneous, overlapping or distinct. For example, a first component can be included in the medium at the outset of mesoderm induction and a second component can be added to the medium during the mesoderm induction phase.

The term “cardiac induction medium” can Include a culture medium that supports induction of cardiac progenitor cells from mesoderm cells, such as for example StemPro-34 minimal media comprising for example a WNT inhibitor, optionally IWP2, VEGF and/or an optionally activin/nodal inhibitor, optionally SB-431542. Depending on the desired cell type, the cardiac induction medium may also comprise a BMP component, retinoic acid, a FGF Inhibitor or a FGF component. One embodiment of a cardiac induction medium (also referred to as standard cardiac induction media) is StemPro-34 minimal media containing 0.5 μM IWP2, 10 ng/ml VEGF, and optionally 5.4 μM SB-431542. Other minimal media that can be used include MesoFate™ (Stemgent) and RPMI (ThermoFisher and other companies).

The term “cardiac induction phase” can be used to describe the time period in which mesoderm cells are induced to differentiate into cardiac progenitor cells when cultured with cardiac induction medium and are treated for example with BMP component and RA as well as optionally a FGF inhibitor or FGF component and/or other components that result in cardiovascular progenitor cells. The treatments can be simultaneous, overlapping or distinct. For example, a first component can be comprised in the medium and a second component can be added to the medium during the cardiac induction phase.

The term “basic medium” can include a culture medium that supports growth of cardiovascular progenitor cells and cardiomyocytes comprising a minimal media such as StemPro 34 (ThermoFisher), MesoFate™ (Stemgent), RPMI (ThermoFisher and other companies), and for example VEGF. An example of a basic medium is provided in Example 1.

The term “basic phase” can be used to refer to the time period cardiovascular progenitor cells are cultured with basic medium and are treated with VEGF and/or other components that result in cardiomyocytes. The treatments can be simultaneous, overlapping or distinct.

The term “incubating” can Include any in vitro method of maintaining and/or propagating a population of cells, including monolayer, bead, flask, or 3D cultures, optionally where ambient conditions are controlled as In an incubator and optionally involving passaging of cells. In steps that involve incubating the cells with one or more components, the components can be added simultaneously, at different times, for overlapping periods or for distinct periods. A factor can be added to the medium after the cells have started incubating in for example an induction medium or the factor can be added to the medium before the medium is added to the cells. Further, cells may be washed between incubations, for example to reduce the level of a component from a previous incubation.

The term “culturing” can include any in vitro method of maintaining and propagating a population of cells at least through one ceil division, including monolayer, bead, flask, or 3D cultures, optionally where ambient conditions are controlled as in an incubator.

The term “enriched for” as used herein means comprising at least 50%, at least 60%, or at least 70% up to 100% of the cell type which is enriched. In one embodiment, enrichment is measured in a day 20 culture using a method as described herein.

The term “subject” as used herein includes al members of the animal kingdom including mammals, and suitably refers to humans.

The terms “treat”, “treating”, “treatment”, etc., as applied to a cell, include subjecting the cell to any kind of process or condition or performing any kind of manipulation or procedure on the cell.

The term “treatment” as used herein as applied to a subject, refers to an approach aimed at obtaining beneficial or desired results, including clinical results and includes medical procedures and applications including pharmaceutical or other product interventions. In one embodiment treatment refers to administration of a product for the purposes of engraftment. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival If not receiving treatment

As used herein, the term “heart failure” refers to a condition in which a subject's heart is unable to pump sufficiently to maintain suitable blood flow in the subject's body. A subject “at risk of heart failure” refers to a subject having one or more characteristics known to precede heart failure. For example, a subject at risk of heart failure may have or have had coronary artery disease, previous myocardial infarction (heart attack), high blood pressure, atrial fibrillation, valvular heart disease, excess alcohol use, tobacco use, obesity, sleep apnea, infection (viral and/or bacterial), cardiomyopathy, myocarditis, congenital heart defects. arrhythmias, and/or other diseases such as, but not limited to, diabetes, hyperthyroidism, hypothyroidism, hemochromatosis and/or amyloidosis.

As used herein, the terms “myocardial infarction” and “MI”, refers to an event in which blood flow decreases or stops to a part of the heart, thereby causing death to cardiomyocytes, due to lack of oxygen supply (ischemia), resulting in damage to the heart muscle.

As used herein, the terms “administering”, “introducing” and “transplanting” and are used interchangeably in the context of delivering cells into a subject, by a method or route which results in at least partial localization of the introduced cells at a desired site.

The term “pluripotent stem cell” or “PSC” as used herein refers to a cell with the capacity, under different conditions, to differentiate into any one of the cell types characteristic of the three germ cell layers, and includes embryonic stem cells and induced pluripotent stem cells. Pluripotent cells are characterized by their ability to differentiate to more than one cell type using, for example, a nude mouse teratoma formation assay. Pluripotency is also evidenced by the expression of embryonic stem (ES) cell markers. As used herein, pluripotent stems can include induced pluripotent stem cells (iPSC) and embryonic stem cells (ESC).

In an embodiment, the term “embryonic stem cells” excludes stem cells involving destruction of an embryo such as a human embryo.

As used herein, the terms IPSC“and induced pluripotent stem cell” are used interchangeably and refer to a pluripotent stem cell artificially derived (e.g., induced or by complete reversal) from a non-pluripotent cell, typically an adult somatic cell, for example, by inducing expression of one or more genes (including, for example, POU4F1/OCT4 (Gene ID; 5460) in combination with, but not restricted to, SOX2 (Gene ID; 6657), KLF4 (Gene ID; 9314), cMYC (Gene ID; 4609), NANOG (Gene ID; 79923), LIN28/LIN28A (Gene ID; 79727)).

Cardiomyocytes prepared, enriched, or isolated by a method of the invention are derived from pluripotent stem cells. For example, a patient's cells may be genetically modified prior to use through introduction of genes that may control their state of differentiation prior to, during or after their exposure to differentiation factors described herein. Pluripotent stem cells suitable for use in methods described herein, which are derived from a patient's own tissue enhances compatibility of differentiated tissue grafts derived from the stem cells with the patient.

The term “embryonic stem cell” is used to refer to the pluripotent stem cells of the inner cell mass of the embryonic blastocyst (see, for example, U.S. Pat. Nos. 5,843,780, 6,200,806). Such cells can also be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, responsiveness to particular culture conditions, and the like.

Pluripotent stem cells, as used herein, may also be genetically modified through introduction of vectors expressing a selectable marker under the control of a stem cell specific promoter, such as Oct-4, or of genes that may be upregulated to Induce cardiomyocyte differentiation. The stem cells may be genetically modified at any stage with markers or genes so that the markers or genes are carried through to any stage of culturing. The markers may be used to purify or enrich the differentiated or undifferentiated stem cell populations at any stage of culture.

The term “pharmaceutically acceptable carrier” as used herein includes essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy) propyl) N,N,N-trimethylammonium chloride (DOTMA), diolesylphosphotidyl-ethanolamine (DOPE), and liposomes. Such compositions should contain a therapeutically effective amount of the compound(s), together with a suitable amount of carrier so as to provide the form for direct administration to the subject.

In understanding the scope of the present disclosure, the term “concentration” as used herein means a final concentration of a substance such as for example BMP4, Activin A, retinoic acid in a medium. Unless indicated otherwise, the concentration is based on a weight/volume ratio.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and “the” include plural referents unless the content clearly dictates otherwise. The term “about” means plus or minus 0.1 to 50%, 5-50%, or 10-40%, preferably 10-20%, more preferably 10% or 15%, of the number to which reference is being made.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### Aspects and Embodiments

In an aspect, there is provided a method of producing a population of cardiomyocytes enriched for atrial cardiomyocytes, the steps comprising: i. incubating pluripotent stem cells in a medium suitable to generate aggregates and/or embryoid bodies, ii. further Incubating the stem cells in a medium suitable for mesoderm induction, wherein said medium at least includes a BMP component, optionally BMP4, and an activin component, optionally Activin A, wherein the BMP component to the activin component is provided in a ratio of 3:2; iii. further adding a retinoic acid component to the cells, said addition of retinoic acid added during the mesoderm induction or cardiovascular specification stage; iv. Continue growth of said cells in suitable medium(s) to generate a population of cardiomyocytes, wherein said population of cardiomyocytes is enriched for atrial cardiomyocytes. In some embodiments the ratio of BMP to activin is 1.5:1.0 (or 3:2).

In some embodiments, said BMP component is BMP4, the activin component is Activin A, the concentration of BMP4 Is 3 ng/ml and the concentration of Activin A is 2 ng/ml. In some embodiments, said retinoic acid component is trans retinoic acid and is added in a concentration of between 50 nm and 5 μM. In some embodiments, said retinoic acid component is added at a concentration of 500 nM.

In some embodiments, the BMP component and the Activin component are added at day 1 of the process. In some embodiments, the retinoic add component is added at day 3 of the process. In some embodiments, additional BMP component is not added to the medium at day 3 of the process.

In some embodiments, an FGF inhibitor is excluded from the medium at day 3 of the process. In some embodiments, the cells produced by the process are utilized in an in vitro assay to screen for cardiac texicity that may be caused by potential therapeutic compounds.

In an aspect, there is provided an isolated population of cardiomyocytes enriched for atrial cardiomyocytes comprising at least or about 50% of atrial cardiomyocytes, at least or about 60% of atrial cardiomyocytes, at least or about 70% of atrial cardiomyocytes, at least or about 80% of atrial cardiomyocytes, or at least or about 90% of atrial cardiomyocytes, preferably obtained according to the method described herein. In an aspect, there is provided a method of producing a population of cardiomyocytes enriched for ventricular cardiomyocytes, the steps comprising: i. incubating pluripotent stem cells in a medium suitable to generate aggregates (embryoid bodies), ii. incubating the aggregated stem cells in a medium suitable for mesoderm induction, wherein said medium at least includes a BMP component, optionally BMP4, and an activin component, optionally Activin A, wherein the concentration of the activin component is greater than the concentration of the BMP component; Ili. continue growth of said cells in suitable medium(s) to generate a population of cardiomyocytes, wherein said population of cardiomyocytes is enriched for ventricular cardiomyocytes. In some embodiments that ratio of BMP to activin is about 0.3:1.0, about 0.5:1.0 (or 1:2) or about 0.8:1.0.

In some embodiments, the concentration of BMP component and/or the Activin component are determined by measuring for the level of CD235a and comparing this to the level of RALDH2.

In some embodiments, the concentration of the Activin component is chosen on the basis of the concentration which preferentially results in more CD235a expressing mesoderm cells as compared with RALDH2 expressing mesoderm cells, and the BMP component is added to achieve a lower concentration than the concentration of the Activin component. In some embodiments, the BMP component is added to achieve optimal cardiogenesis from the induced mesoderm.

In some embodiments, said BMP component is BMP4, the activin component is Activin A, the concentration of BMP4 is between 3-20 ng/ml, the concentration of the Activin A is between 4-20 ng/ml, and the concentration of the Activin A is greater than the concentration of the BMP4. In some embodiments, the concentration of BMP4 is 10 ng/ml and the concentration of Activin A is 12 ng/ml.

In an aspect, there is provided an isolated population of cardiomyocytes: enriched for ventricular cardiomyocytes comprising at least or about 30% of ventricular cardiomyocytes, at least or about 40% of ventricular cardiomyocytes, at least or about 50% of ventricular cardiomyocytes, at least or about 60% of ventricular cardiomyocytes, at least or about 70% of ventricular cardiomyocytes, at least or about 80% of ventricular cardiomyocytes, or at least or about 90% of ventricular cardiomyocytes, preferably obtained according to the method described herein. In an embodiment, the isolated population of cardiomyocytes enriched for ventricular cardiomyocytes is essentially free of pacemaker cells. In a preferred embodiment, the isolated population of cardiomyocytes enriched for ventricular cardiomyocytes is devoid of pacemaker cells.

An isolated population of cardiomyocytes according to the invention may be used in a method for screening for potential cardiac toxicity of potential therapeutic active agents for use in treating cardiovascular and any other disorders. For example, they provide a source of cells that can be used in drug screens for cardiovascular applications; they provide a source of cells that can be used for therapeutic purposes—to restore cardiac function; to repair the ischemic heart and/or to regenerate the coronary vasculature; they can be used for tissue engineering purposes where components of the heart or the coronary vasculature are required; and they may serve as a research tool for the study of cardiovascular development and disease. An isolated population of cardiomyocytes used for the screening of active agents, according to methods of the invention may, for example, include cardiomyocyte populations enriched for ventricular cardiomyocytes. Such ventricular cardiomyocyte populations include, optionally, populations which are essentially free of pacemaker cells, or devoid of pacemaker cells. An isolated population of cardiomyocytes used to scree active agents, according to methods of the invention, may also include a population enriched for atrial cardiomyocytes. Such methods for screening or evaluating the potential cardiac toxicity of a test compound or agent, involve exposing a population of cardiomyocytes according to the present invention to a compound to be tested for cardiotoxicity. Effects to evaluated include changes in the viability, contractility, membrane electric potentials and/or other functionalities of the cells.

Cardiomyocyte and cardiomyocyte progenitor cell populations produced using methods of the invention that may be used for transplantation, cell therapy or gene therapy. For example, the invention provides differentiated cells produced using methods of the invention that may be used for therapeutic purposes, such as in methods of treating a subject in need of cardiac repair. For example, therapeutic repair may involve restoring, in full or in part, cardiac function in a subject in need of cardiac repair, such as a subject suffering from a heart disease or condition.

Another aspect of the invention is a method of treating or preventing a cardiac disease or condition. Cardiac disease Is typically associated with decreased cardiac function and includes conditions such as, but not limited to, myocardial infarction, cardiac hypertrophy and cardiac arrhythmia. In this aspect of the invention, the method includes introducing into a subject in need of cardiac repair, isolated differentiated ventricular cardiomyocyte cells of the invention and/or cells capable of differentiating into ventricular cardiomyocyte cells. The isolated cardiomyocyte cells may be transplanted into damaged cardiac tissue of a subject. Ideally, the method results in the restoration of some or all cardiac function in a patient.

In an aspect, there is provided a method of treating a subject with heart failure, comprising administering to the subject the population of ventricular cardiomyocytes described herein. In some embodiments, said subject is suffering from a myocardial infarction. In some embodiments, the myocardial infarction is in the ventricle of the patient and the population is as described herein.

In an aspect, there is provided the population of ventricular cardiomyocytes described herein, for use in the treatment of a subject with heart failure or at risk of heart failure. In an aspect, there is provided use of the population of ventricular cardiomyocytes described herein, in the preparation of a medicament for the treatment of a subject with heart failure or at risk for heart failure.

In yet another aspect of the invention there is provided a method of repairing cardiac tissue, the method including introducing an isolated ventricular cardiomyocyte or cardiac progenitor cell of the invention and/or a cell capable of differentiating into a ventricular cardiomyocyte cell when treated using a method of the invention into damaged cardiac tissue of a patient.

The patient may be suffering from a cardiac disease or condition. In the method of repairing cardiac tissue of the present invention, the isolated cardiomyocyte cell may be transplanted into damaged cardiac tissue of a patient. Ideally, the method results in the restoration of at least some cardiac function in a patient.

In one embodiment, ventricular cardiomyocytes disclosed herein are administered to a subject during the acute phase after myocardial infarction or during the chronic stage of heart failure. Colls are administered to the site of damage in the ventricle either by direct injection or catheter-based delivery. Calls may be formulated together with pharmaceutically acceptable carriers, hydrogels or scaffolds, for example, to aid in placement, survival and/or engraftment of the cells in the tissue. Cell dosage ranges may include, for example, from about 0.5 billion to 2 billion cells per dose. The cells may be administered to the subject in single or multiple doses, at one or more point in time in order to treat the subject.

The present invention preferably provides a myocardial model for testing the ability of stems cells that have differentiated into cardiomyocytes or cardiac progenitors using methods of the invention to restore cardiac function. In order to test the effectiveness of cardiomyocyte transplantation in vivo, it is important to have a reproducible animal model with a measurable parameter of cardiac function. The parameters used should clearly distinguish control and experimental animals [see for example in Palmen St al. (2001), Cardiovasc. Res. 50, 516-524] so that the effects of transplantation can be adequately determined. PV relationships are a measure of the pumping capacity of the heart and may be used as a read-out of altered cardiac function following transplantation.

In an aspect, there is provided a process for detecting atrial mesoderm in a population of cells, comprising detecting RALDH2, wherein a presence of RALDH2 is indicative of atrial mesoderm. In an aspect, there is provided a process for detecting ventricular mesoderm in a population of cells, comprising detecting CD235a and/or CYP26A1, wherein a presence of CD235a and/or CYP26A1 is indicative of ventricular mesoderm.

Methods of the invention for identifying atrial or ventricular mesoderm on the basis of ALDH, preferably RALDH2, and/orCD235a and/or CD235b, and/or CYP26A1 expression, respectively are provided. More particularly, they can be used for identification of secreted factors produced by the mesodermal cell which influence cardiomyocyte proliferation, survival, function and differentiation of atrial or ventricular cell populations. For example, methods of the invention for identifying atrial or ventricular cardiomyocyte populations provide systems to both understand atrial and ventricular mesoderm differentiation at the molecular level and to identify new drug targets(e.g., signaling pathways) that modulate differentiation.

According to one or more of the embodiments disclosed herein Retinoic ad (RA) specifies atrial cardiomyocytes within a specific developmental time window and the application of RA to mesoderm from day 3-5 specifies atrial cardiomyocytes. RA concentration range: 50 nM-5 uM. RA sources: all-trans RA, retinoic receptor (RAR) agonists (AM580 for—alpha, AC55649 for -β, CD437 for-γ) Agonist concentrations: 3-300 nM for AM580; 0.025-2.5 uM AC55649; 0.05-5 uM CD437.

RALDH2 (Retinaldehydrogenase, or Aldefluor) is a marker for atrial mesoderm. The proportion of RALDH2⁺ cells is monitored by using the aldefluor assay for optimizing atrial differentiation. Days of analysis: day 2-6.

The early mesoderm inductions using Activin A and BMP4 at day 1 determine the proportion of RALDH2⁺ mesodermal cells at day 4. Induction conditions are low BMP (1-5 ng/ml BMP) and low Activin A (0.1-4 ng/ml), most commonly used 3 ng/ml BMP/2 ng/ml Activin A (3B12A).

The functionality of RALDH2 is shown by the treatment with retinol (ROH) at day3-5, which is sufficient to induce an atrial phenotype. (Retinol is converted by RALDH2 into RA, RA than specifies the atrial phenotype). Glycophorin A (CD235a) is a marker for ventricular mesoderm. CD235a is expressed exclusively on the ventricular mesoderm and absent on the RALDH2⁺ atrial mesoderm. The CD235a⁺ cells do not express RALDH2. The CD235a⁺ cells express CYP26A1, an enzyme that degrades RA, to antagonize RA signaling and assure the establishment of a ventricular phenotype. Days of analysis: day 2-6.

The early mesoderm inductions using Activin A and BMP4 at day1 determine the proportion of CD235a⁺ mesodermal cells at day 4. Induction conditions are high BMP (5-20 ng/ml BMP), and high Activin (6-20 ng/ml), most commonly used 10 ng/ml BMP/12 ng/ml Activin A (10B/12A). Treatment of the CD235a⁺ cells with retinol (ROH) at day3-5 is NOT sufficient to induce an atrial phenotype. (These cells are not able to convert retinol into RA, therefore the cells develop into a ventricular phenotype). The CD235a⁺ cells are giving rise to populations highly enriched in MLC2V⁺ ventricular cardiomyocytes.

Ventricular and atrial cardiomyocytes are derived from two distinct mesodermal subpopulations. The ventricular differentiation is monitored by the emergence of day4 CD235a+ cells and day20 MLC2V⁺/CTNT⁺ cells. The atrial differentiation is monitored by the emergence of day4 AF+ cells and day20 MLC2v⁻/CTNT⁺ cells. The day20 population derived from the ventricular mesoderm (10B/12A) contains a higher proportion of MLC2v⁺ ventricular cardiomyocytes than those derived from the atrial mesoderm (3B/2A).

Gene expression analysis and single cell patch clamp analysis showed that the day20 population generated by RA treatment from the atrial mesoderm (3B/2A+RA) contains a higher proportion of atrial cardiomyocytes than the day20 population generated by RA treatment from the ventricular mesoderm (10B/12A+RA)

The proper mesoderm subpopulations need to be specified to enrich for the desired cardiomyocyte subtypes. Improved protocol for the specification of ventricular cardiomyocytes for cell replacement therapy after myocardial infarction. The CD235a⁺ ventricular mesoderm (10B/12A) is giving rise to populations highly enriched for MLC2v⁺ ventricular cardiomyocytes devoid of pacemaker cells. This results in lower spontaneous beating rates compared to other heterogeneous cardiomyocyte populations.

These are desirable characteristics for cell replacement therapies after myocardial infarction because mixed cell populations that contain contaminating pacemaker cells and have fast spontaneous beating rates can cause life threatening arrhythmias. We propose that our new protocol for specification of ventricular cardiomyocytes is superior to previous protocols that generated mixed populations of ventricular and pacemaker cells.

### Examples

### Methodologies and Results

Human pluripotent stem cell lines can be cultured as previously described (e.g. Kennedy et al., 2007). For differentiation into the cardiac lineage, an established protocol such as that described in Kattman et al., 2011) can be used. Various modifications to the procedures are possible including those as described WO2016131137. In one embodiment 80% confluent hPSCs cultures can be dissociated into single cells, suspended in StemPro-34 Media containing 1 ng/ml BMP4 and 10 μM ROCK inhibitor and incubated for 18 hours on an orbital shaker to generate embryoid bodies (EBs). The next day (day 1 of differentiation) the EBs can be transferred to mesoderm induction media: StemPro-34 containing a set concentration of BMP4, and a set concentration of Activin A as further described herein, as well 5 ng/ml bFGF. At day 3 of differentiation the EBs can be washed once using IMDM and suspended In cardiac induction media: in one embodiment cardiac induction media can include StemPro-34 containing 0.5 μM IWP2, 10 ng/ml VEGF, and optionally 5.4 μM SB-431542 (SB, Activin/Nodal/TGFβ inhibitor). Cardiac induction media can also optionally include retinoic acid (RA), or an RA component as further described herein.

Retinoic acid signaling specifies atrial-Ike cardiomyocytes from hESCs

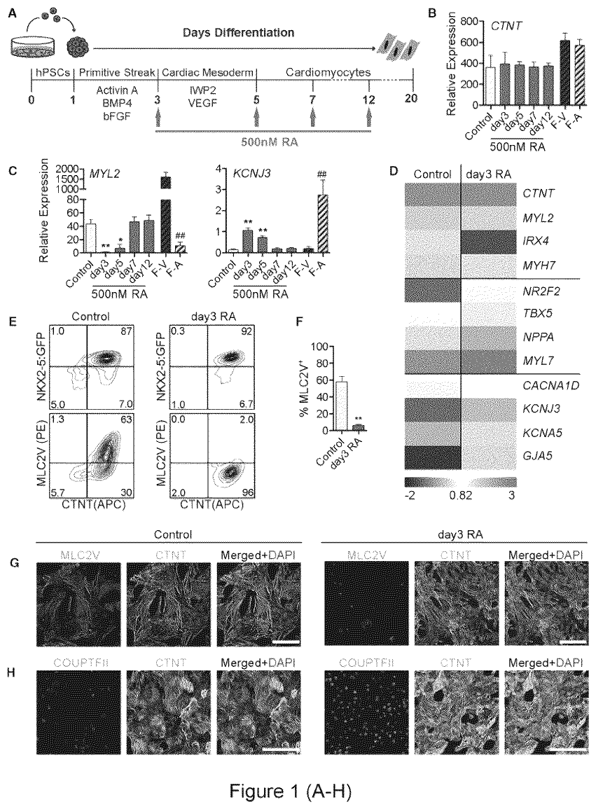

To determine if retinoic acid signaling can specify an atrial fate in hPSC-derived cardiogenic populations generated with our embryoid-body (EB)-based protocol, all trans retinoic acid (RA) was added to the differentiation cultures at 4 different time points that represent the following developmental stages: mesoderm induction (day 3), cardiovascular specification (day 5), cardiac progenitor development (day 7) and emergence of contracting cardiomyocytes (day 9) (Kattman et al., 2011) (FIG. 1A). The HES3 NKX2-5: GFP reporter hESC line was used for these experiments to allow us to monitor and quantify cardiovascular development and to isolate GFP+ cardiomyocytes. At day 20 of culture, GFP+SIRPA+CD90− cardiomyocytes were isolated from the differentiated populations and analyzed by RTqPCR for expression of genes indicative of atrial and ventricular development. (FIGS. 1B-D and 8B-E).

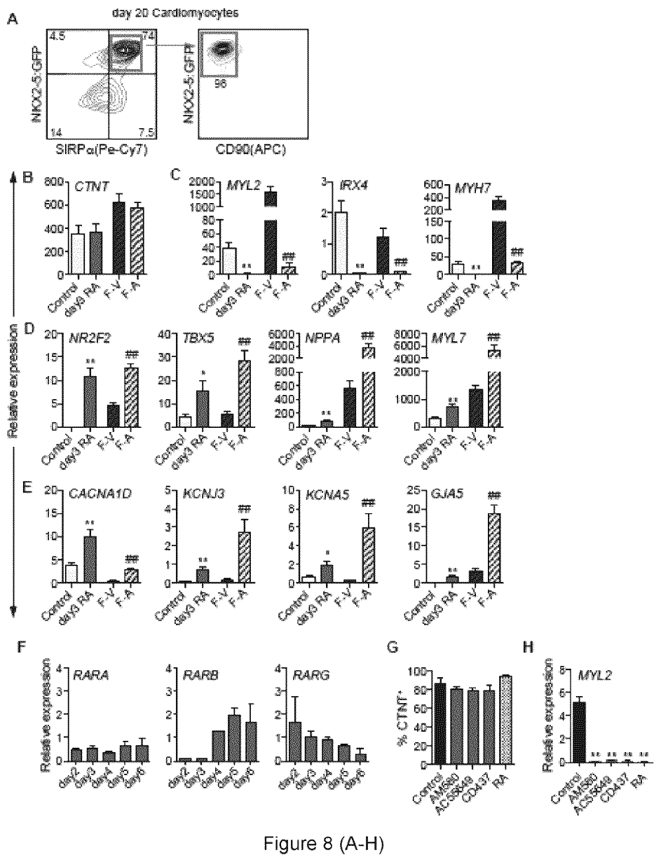

None of the RA treatments significantly altered the levels of expression of the pan-cardiomyocyte marker CTNT, Indicating comparable cardiomyocyte content in the different populations (FIG. 1B). Addition of RA at days 3 and 5 resulted in a significant reduction in expression of the ventricular-specific gene MYL2 and an upregulation of the atrial ion channel gene KCNJ3 (FIG. 1C) suggesting a change in cardiomyocyte fate in the day 20 populations. Interestingly, addition of RA at later stages (days 7 and 12) had no effect on expression of these genes. Analyses of additional chamber-specific markers showed that cardiomyocytes generated from day 3 RA-treated mesoderm also expressed lower levels of the ventricular markers IRX4 and MYH7 than the non-treated group, whereas the reverse pattern was observed for the atrial markers NR2F2, TBX5, NPPA, and MYL7 and atrial-specific ion channels CACNA1D, KCNA5, and GJA5 (FIGS. 1D and 8C-E). Analyses of control fetal tissues verified the atrial and ventricle expression patterns of these different genes. Flow cytometric and immunostaining analyses of cardiomyocyte populations generated from day 3 RA-treated mesoderm confirmed the qRT-PCR expression patterns, and they showed a dramatic reduction in the proportion of MLC2V⁺ cells and a much higher frequency of COUPTFII⁺ cells in the population generated from day 3 RA-treated mesoderm comparted to the one generated from the non-treated control mesoderm (FIGS. 1E-H).

Taken together, these findings strongly suggest that RA signaling induces a fate change in hPSC cardiogenesis, promoting the development of atrial cardiomyocytes at the expense of the ventricular lineage. Additionally, they show that this effect of RA is restricted to an early developmental window, between days 3 and 5 of differentiation, corresponding to the mesoderm state of differentiation.

To further characterize the RA response, we next analyzed populations between days 2 and 6 of differentiation for expression of the 3 RA receptor (RAR) isoforms; RAR-alpha, -β, -γ (RARA, RARB, and RARG in FIG. 8F, respectively). All three were expressed during the responsive stage, suggesting that the RA response may be mediated through all of them (FIG. 8F). To test this, we added the receptor-specific agonists AM580 for alpha, AC55649 for β or CD437 for γ in place of RA to the day 3 cultures. Addition of each of the agonists led to a reduction of MYL2 expression in day 20 CTNT⁺ populations, suggesting that signaling through all receptor isoforms can mediate the change in fate (FIGS. 8G and 8H).

In some embodiments RA can be added in a concentration of about 0.05 μM to a concentration of about 5 μM. In one embodiment the concentration of RA is 500 nM (0.5 μM). In one embodiment the concentration of RA added is between 0.05 μM and 0.01 μM. In one embodiment the concentration of RA added is between 0.01 μM and 0.1 μM. In some embodiments an RA component is added. In some embodiments the RA component is a retinoic acid receptor (RAR) agonist. In some embodiments the RAR agonist is an agonist against the alpha receptor. In some embodiments the RAR agonist is AM580. In some embodiments the AM580 RAR agonist is added in a concentration of about 3 nM to about 300 nM. In some embodiments the RAR agonist is an agonist against the beta receptor. In some embodiments the RAR agonist is AC55649. In some embodiments the AC55649 is added in a concentration of about 0.025 μM to 2.5 μM. In some embodiments the RAR agonist is an agonist against the gamma receptor. In some embodiments the RAR agonist is CD437. In some embodiments the CD437 RAR agonist is added in a concentration of about 0.05 μM to about 5 μM.

RALDH2 and CYP26A1 expression identifies mesoderm subpopulations

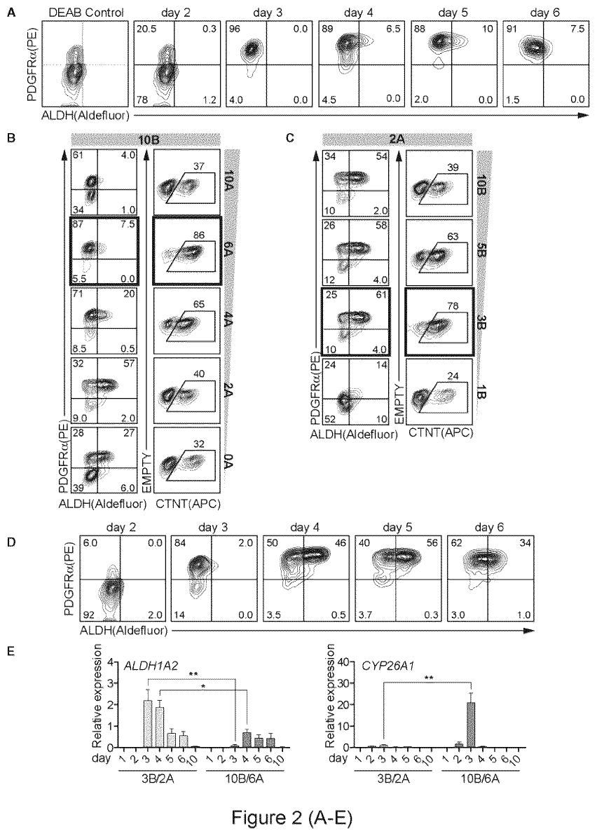

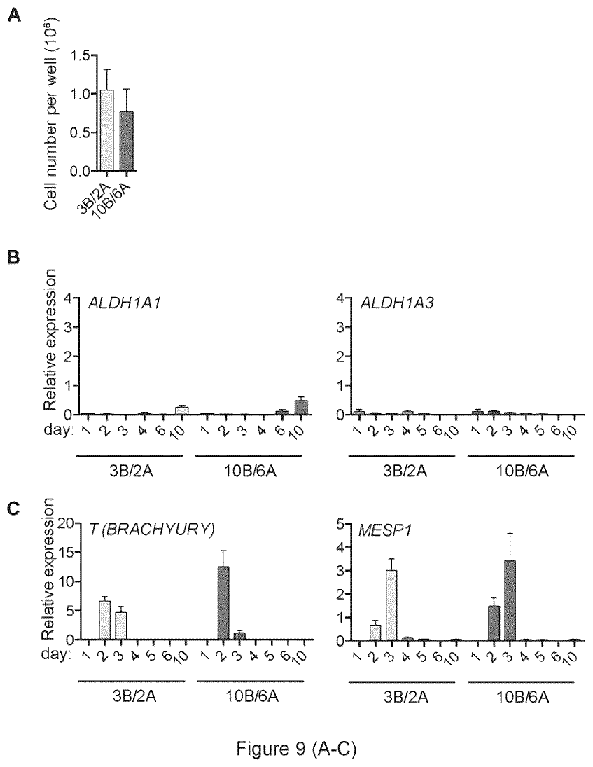

If specification of atrial fate is mediated via autocrine RA signaling, the mesoderm population that gives rise to these cardiomyocytes should display RALDH activity. To test this we analyzed PDGFRalpha mesoderm induced with our standard conditions (10 ng/mL BMP4 and 6 ng/mL Activin A (10B/6A) on different days, using the adefluor assay that detects the activity of all aldehyde dehydrogenases, (ALDHs), including the three retinaldehyde dehydrogenases, RALDH1, -2. and -3 (Jones et. al., 1995). These analyses revealed the presence of a small ALDH+PDGFRalpha+ population at days 4 and 5 of differentiation (FIG. 2A), suggesting that a subpopulation of mesoderm at these stages may have the capacity to synthesize RA. In an attempt to increase the size of the ALDH⁺PDGFRalpha⁺ population, we tested the effect of varying the concentrations of Activin A and BMP4 during the mesoderm induction step. Reducing the amount of Activin A In the presence of a constant concentration of BMP4 (10 ng/mL) led to a substantial increase in the size of the ALDH⁺ PDGFRalpha⁺ population at day 4 of differentiation (FIG. 2B). However, this increase was associated with a decrease in the proportion of CTNT⁺ cardiomyocytes generated, suggesting that these changes promoted a non-cardiogenic fate. As we have previously shown that the ratio of Activin A and BMP4 signaling is important for maintaining optimal cardiogenic potential (Kattman et al., 2011), we next varied the concentration of BMP4 in the presence of the amount of Activin A (2 ng/mL) that induced the largest ALDH⁺ PDGFRalpha⁺ population. Reducing the BMP4 concentration from 10 to 3 ng/mL (3B/2A) did not influence the size of the ALDH⁺ PDGFRalpha⁺ population, but it did increase the frequency of CTNT⁺ cells generated at day 20 (FIG. 2C). Comparable cell numbers were obtained from the 3B/2A and 10B/6A cultures, indicating that the manipulations did not significantly impact total cardiomyocyte output (FIG. 9A).

Analyses of cultures induced with 3B/2A revealed the emergence of a large PDGFRalpha⁺ mesoderm population at day 3 of differentiation, followed by the development of an ALDH⁺ PDGFRalpha⁺ population at day 4 (FIG. 2D). The size of the ALDH⁺PDGFRalpha⁺ population increased until day 5 and then started to decrease at day B. Molecular analyses showed that expression of RALDH2 (ALDH1A2) increased sharply between days 2 and 3 of differentiation, and then it declined over the next 7 days in the group induced with 3B/2A (FIG. 2E). The 10B/6A-induced cells ex-pressed significantly lower levels of ALDH1A2 at days 3 and 4, consistent with the smaller proportion of ALDH⁺ cells in this group. The expression levels of other RALDH isoforms (ALDH1A1 and ALDH1A3) were markedly lower than those of ALDH1A2, and they did not differ between the two populations (FIG. 9B). T (BRACHYURY) and MESP1 showed similar temporal expression patterns in both the 10B/6A- and 3B/2A-induced populations, indicating that the kinetics of mesoderm induction were not dramatically different between the two groups (FIG. 9C). In the developing embryo, the boundaries of RA activity and the duration of signaling are established by a balance between localized agonist synthesis and degradation (Cunningham and Duester, 2015; Rydeen and Waxman, 2014). To determine if this balance is at play in the hPSC differentiation cultures, we next analyzed the two populations for expression of CYP26A1, a member of cytochrome P450 family enzyme responsible for RA degradation. These analyses revealed a striking difference between the two groups, with the day 3 10B/6A-induced cells showing dramatically higher expression levels than any other 10B/6A- or 3B/2A-induced population (FIG. 2E). Collectively, these findings support the interpretation that combinations of 38/2A and 100B6A induce different mesoderm populations distinguished by expression of ALDH1A2 and CYP26A1.

Retinol specifies ALDH+ mesoderm to an atrial fate.

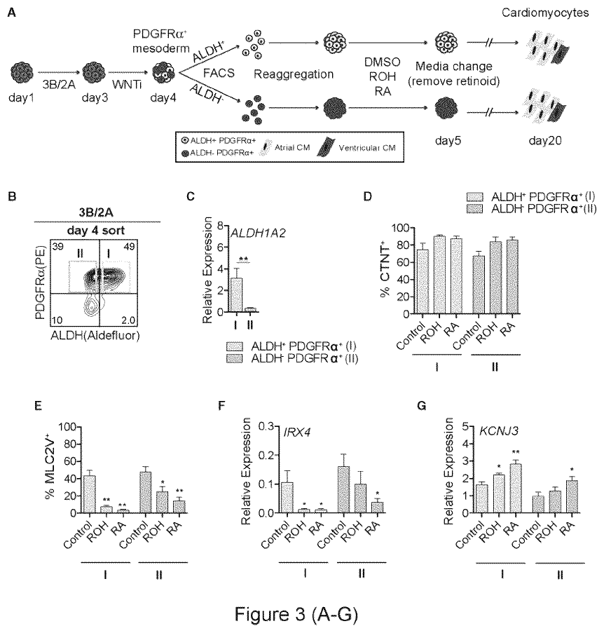

To determine if the ALDH⁺ cells can synthesize RA, the ALDH⁺ PDGFRalpha⁺ and ALDH⁻ PDGFRalpha⁺ fractions were isolated from the day 4 3B/2Ainduced population, and the cells were cultured as aggregates in retinol (ROH), RA, or DMSO (control) for 24 hr (FIGS. 3A and 3B). ALDH1A2 expression segregated to the ALDH⁺ fraction, confirming the validity of aldefluor-based sorting strategy for isolating RALDH2-expressing cells (FIG. 3C). Following an additional 15 days of culture, all groups contained a high proportion of CTNT⁺ cells, demonstrating efficient cardio-myocyte differentiation (FIG. 3D). The untreated controls generated cardiomyocyte populations that contained MLC2V⁺ cells and expressed IRX4, demonstrating that, in the absence of RA signaling, the 3B/2A-induced mesoderm has some ventricular cardiogenic potential (FIGS. 3E and 3F). Following treatment with ROH, the ALD⁺H mesoderm generated an atrial-like cardiomyocyte population that had a lower frequency of MLC2V⁺ cells, lower levels of IRX4 expression, and elevated levels of KCNJ3 expression compared to the untreated control (FIGS. 3E-3G). The expression patterns in the ROH- and RA-treated ALDH⁺ PDGFRalpha⁺-derived populations were similar, strongly suggesting that the ALDH⁺ cells were able to synthesize RA from ROH.

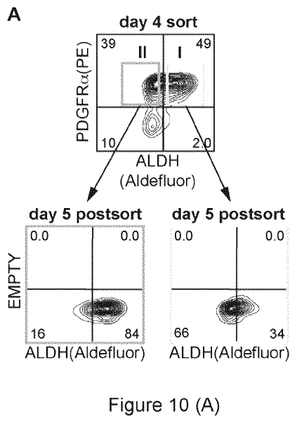

Surprisingly, we observed a similar response to ROH in the ALDH-cells (FIG. 3E-3F) despite their lack of ALDH1A2 expression at the time of isolation (FIGS. 3B and 3C). This response was likely due to the fact that the majority of the ALDH−-derived population became ALDH+ during the 24-hour aggregation culture (FIG. 10A), enabling the cells to respond to ROH. Interestingly, we observed a decrease in aldefluor staining in the ALDH+-derived population over the same 24-hour period, highlighting the dynamic natures of the ALDH activity (RALDH2 expression) within the mesoderm population.

Together, these findings demonstrate that 3B/2A induces ALDH+PDGFRalpha+(RALDH2+) mesoderm that can respond to ROH and generate atrial-like cardiomyocytes, supporting the notion that specification of this fate is mediated via autocrine RA signaling.

CD235a expression marks mesoderm that gives rise to ventricular cardiomyocytes. It is contemplated herein that CD235b may replace CD235a as a marker of mesoderm that gives rise to ventricular mesoderm, at least due to the amino acid sequence similarity and/or identity of the N-terminal region of Glycophorin B and Glycophorin A.

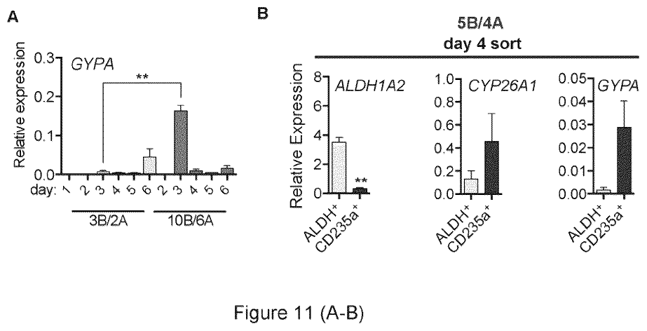

To be able to monitor the development of AYP26A1-expressing mesoderm (VM) that gives rise to ventricular cardiomyocytes, we initiated a search for surface markers on this population that would allow us to distinguish it from the ALDH+ mesoderm. Through a previous screen on an anti-CD antibody array (http://www.ocigc.ca/antibody/), we found that glycophorin A (CD235a) was expressed on a subset of day 5 cardiogenic PDGFRalpha+ cells induced with 10B/6A (data not shown). Analyses of 10B/6A- and 3B/2A-induced populations revealed that CD235a+ population increased within the next 24 hr (>60%) and then declined over the following 48 hr. The small proportion of ALDH⁺ cells detected at day 5 were CD235a⁻, indicating that the ALDH⁺ and CD235a⁺ populations are mutually exclusive. Only a few CD235a⁺ cells were detected at day 4 in the 3B/2A-induced populations. The qRT-PCR analyses revealed an upregulation of GYPA (glycophorin A) expression onday 3 of differentiation in the group induced with 10B/6A. FIG. 11A). The expression levels declined sharply over the next 24 hours and remained low for the duration of the analyses. Only low levels of expression were detected in the 3B/2A-induced populations. Based on these findings, we hypothesize that glycophorin A is expressed on mesoderm that contributes to the ventricular cardiomyocyte lineage.

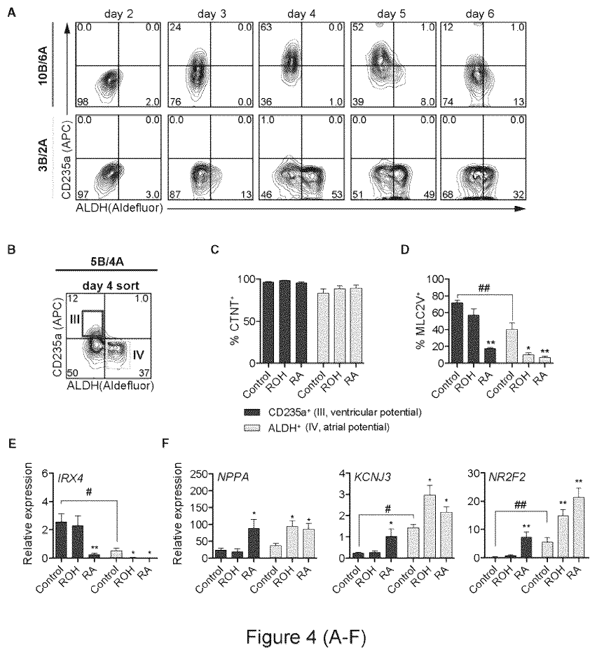

To test the utility of CD235a for the isolation of ventricular pro-genitors, we generated a day 4 population that contained both CD235a⁺ and ALDH⁺ subpopulations using an induction strategy with intermediate concentrations of BMP4 and Activin A (5 ng/mL BMP4 and 4 ng/mL Activin A [5B/4A]) (FIG. 4B). Both the CD235a⁺ALDH⁻ and CD235a⁻ALDH⁺ fractions were isolated and the cells cultured as aggregates. The qRT-PCR analyses of the sorted fractions showed that ALDH1A2 was expressed at higher levels in the CD235a⁻ALDH⁺ cells than in the CD235a⁺ALDH⁻ cells (FIG. 11B). The levels of GYPA and CYP26AI expression were not significantly different between the two, likely due to the fact that the fractions were isolated at day 4, a day beyond the peak expression of these genes. In the absence of ROH and RA, both fractions generated ventricular-like cells (FIGS. 4C-4E). However, the proportion of MLC2V⁺ cells and the expression of IRX4 were higher in the population generated from the CD235a⁺ALDH⁻ mesoderm than in the CD235a⁻ALDH⁺ derivatives. The reverse pattern was observed for the atrial genes KCNJ3 and NR2F2 (FIG. 4F). When cultured in the presence of ROH, the CD235a⁻ALDH⁺ gave rise to an atrial-like cardiomyocyte population characterized by a low frequency of MLC2V⁺ cells; low levels of IRX4 expression; and elevated levels of NPPA, KCNJ3, and NR2F2 expression (FIGS. 4D-4F). The CD235a⁺ALDH⁻ cells by contrast showed no response to ROH, demonstrating an inability to synthesize RA in the absence of ALDH⁻ cells. As expected, both mesoderm populations responded to RA and generated MLC2V⁻ cells. Taken together, these findings demonstrate that CD235a expression marks a mesoderm population with ventricular cardiomyocyte potential that is unable to respond to ROH to generate atrial cells, a characteristic that distinguishes it from the CD235a⁻ALDH⁺ mesoderm. These findings also suggest that the CD235a⁺ and ALDH⁺ mesoderm populations are already patterned to their respective fates, as indicated by the differential expression of the ventricular and atrial genes in the cardiomyocyte populations generated in the absence of RA signaling.

### Optimization of Ventricular Cardiomyocyte Differentiation

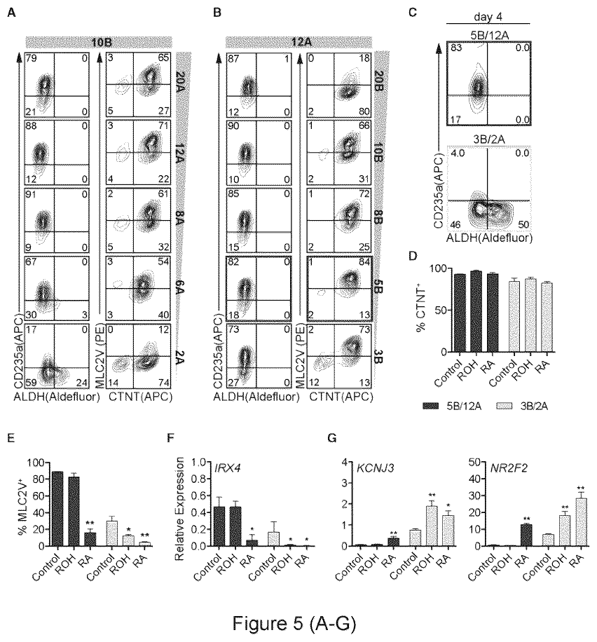

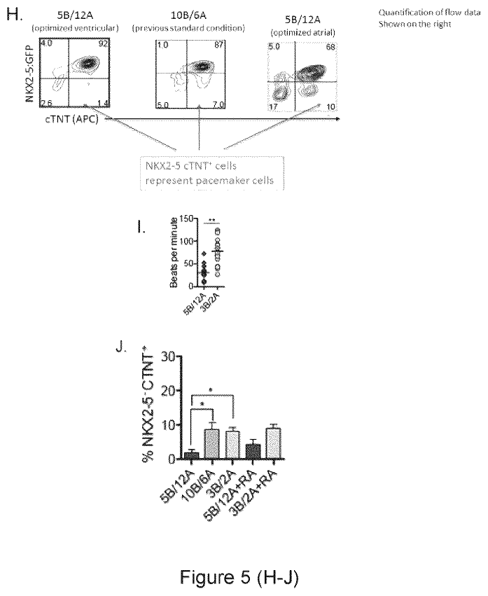

Although induction with 10B/6A favors the development of ventricular cardiomyocytes, the mesoderm generated under these conditions often contains a small proportion of ALDH+ cells and gives rise to CTNT+ populations that contain variable proportions (40% to 60% of MLC2V+ cells. To further optimize ventricular cardiomyocyte development, we monitored the size of the CD235a+ fraction in day 4 EB populations induced with different concentrations of Activin A and BMP4 and compared this to the frequency of MLC2V+CTNT+ cells at day 20 (FIGS. 5A and 5B). Increasing the concentration of Activin A from 2 ng/ml to 12 ng/ml in the presence of a constant amount of BMP4 (10 ng/mL) led to an increase in the size of the day 4 CD235a+ population, the elimination of the ALDH++ population, and an increase in the proportion of MLC2V+CTNT+ cells (FIG. 5A).

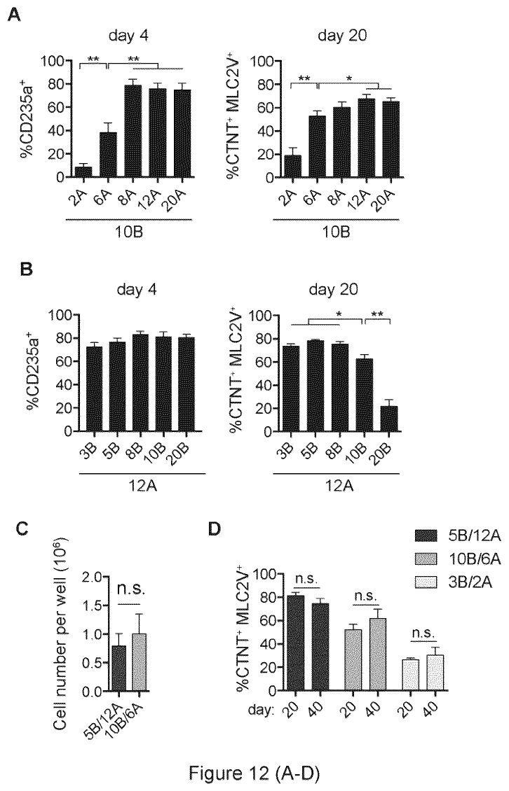

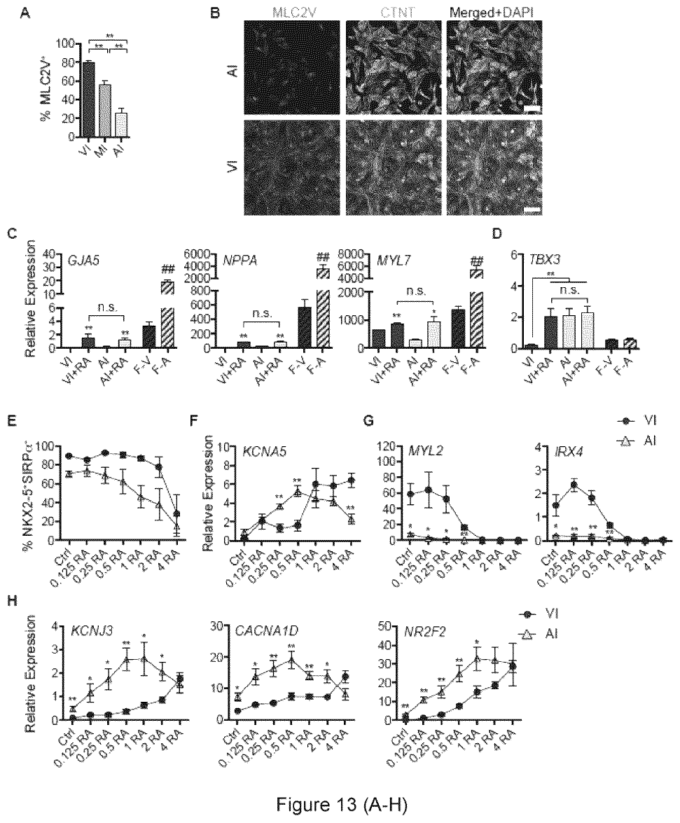

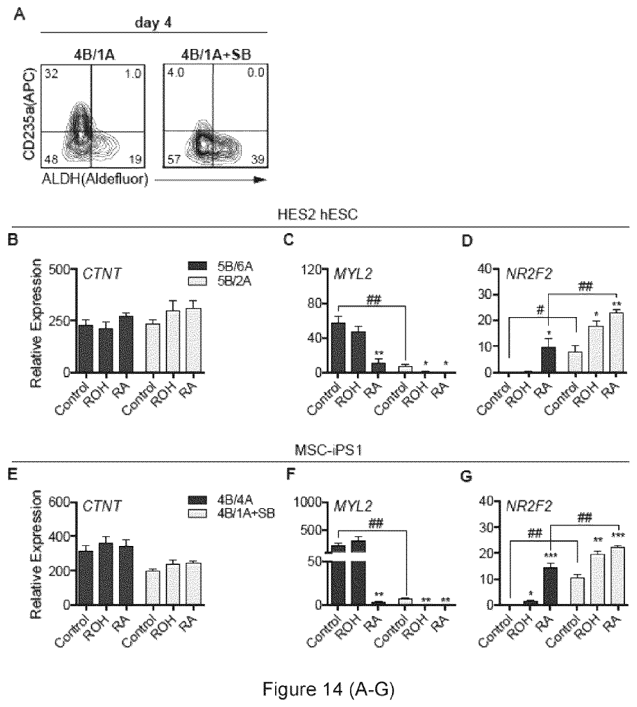

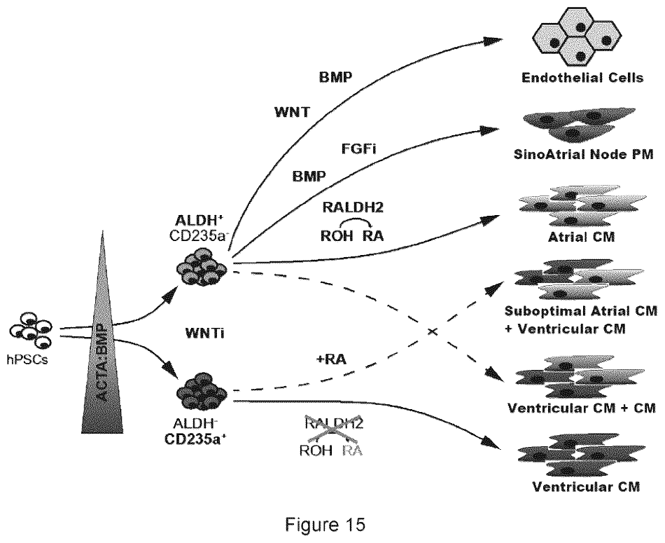

Next, the concentration of BMP4 (3-20 ng/mL) was varied against the amount of Activin A (12 ng/mL) that generated the highest frequency of MLC2V⁺CTNT⁺ cells. Changes in BMP4 concentration had little impact on the size of the CD235a⁺ population, but they did influence ventricular specification. Day 20 populations generated from EBs induced with the highest concentration (20 ng/mL) of BMP4 had the lowest frequency of MLC2V⁺CTNT⁺ cells, whereas EBs induced with a low concentration of BMP4 (5 ng/mL [5B/12A]) generated the highest frequency of these cardiomyocytes (80% ±5%) (FIGS. 58 and 13B). The 5B/12A- and 10B/6A-induced cultures yielded comparable cell numbers, indicating that the enrichment of MLC2V⁺CTNT⁺ cells was obtained without compromising the total cell output (FIG. 12C).

It Is worth noting that the optimal concentration of Activin A and BMP4 is dependent on the activity of the particular lot of cytokine. Given this, these titrations need to be repeated with each new lot of cytokine to determine the optimal concentration. To determine if time in culture could influence the MLC2V+ content of the hPSC-derived cardiomyocyte populations as has been reported (Burridge et al., 2014), we compared day 20 and 40 populations generated from EBs induced with 3B/2A, 10B/6A, and 5B/12A. As shown in FIG. 13B, similar proportions of MLC2V+CTNT+ cardiomyocytes were detected at both time points in each of the populations, demonstrating that extended time in culture did not influence their ventricular content under these conditions. Taken together, these findings indicate that induction of a day 4 CD235a+ population is a pre-requisite for the generation of populations highly enriched in MLC2V+CTNT+ cardiomyocytes. However, they also show that the size of this population is not necessarily predictive of the percentage of MLC2V+ cells at day 20 of culture. The EB population induced with 5B/12 A contained a high proportion of CD235a+ cells and no ALDH+ cells (FIG. 5C) whereas the one induced with 3B/2A had a high frequency of ALDH+ cells and few CD235a⁺ cells. When specified in the absence or presence of ROH or RA (days 3-5) and cultured for an additional 15 days, both populations displayed similar cardiogenic potential as measured by the frequency of CTNT⁺ cells generated (FIG. 5D).

The 3B/2A-induced EBs responded to ROH and generated an atrial-like cardiomyocyte population, characterized by a loss of MLC2V⁺ cells, a reduction in IRX4 expression, and an upregulation of KCNJ3 and NR2F2 expression (FIGS. 5E-5G). In contrast, the 5B/12A-induced EBs did not respond to ROH, consistent with a complete absence of ALDH⁺ cells. As expected, RA treatment was able to induce an atrial-like cardiomyocyte phenotype from this mesoderm.

To determine if the conditions used to optimize ventricular differentiation impacted the proportion of NKX2.5⁻ sinoatrial node pacemaker-like cells (Birket et al., 2015. Protze et al., 2017) normally detected in these cultures, we analyzed the population for the presence of NKX2-5-GFP⁻ cells. As shown in FIG. 5H, the population generated from the optimized 5B/12A-induced EBs contained significantly fewer NKX2-5-GFP-CTNT⁺ cells than those derived from 10B/6A-induced (FIG. 1E) and 3B/2A-induced EBs, indicating a reduced sinoatrial node-like pace-maker cell (SANPLC) content. This decrease in pacemaker content was associated with a significant decrease in spontaneous beating rates of the 5B/12A-induced EBs compared to 3B/2A-induced EBs (FIG. 5I). Consistent with our previous findings (Protze et al., 2017), RA treatment did not influence the proportion of NKX2-5-GFP⁻ cells in the derivative populations (Figure J).

Taken together, these findings show that 5B/12A specifies a subpopulation of mesoderm that contains a high proportion of CD235a⁺ cells and gives rise to populations highly enriched in ventricular cardiomyocytes and devoid of atrial cardiomyocytes and SANPLCs. This subpopulation may also be referred to as ventricular mesoderm.

In some embodiments, optimization of the ventricular differentiation results in enrichment of at least 50%, at least 60%, at least 70%, at least 80%, at least 90%, at least 95% of ventricular cardiomyocytes when measured in a day 20 culture using a method as described herein. In some embodiments, the population is essentially free of pacemaker cells. In some embodiments, the population is devoid of pacemaker cells.

In some embodiments, methods of optimizing ventricular differentiation optimize the generation of the ventricular mesoderm by addition of optimized concentrations of a BMP component and an activin component. In some embodiments the BMP component is BMP4 and the activin component is Activin A. In some embodiments, the BMP4 is added in a concentration of 3 ng/ml to 20 ng/ml. In some embodiments the Activin A is added in a concentration of 4 ng/ml to 20 ng/ml. In a preferred embodiment, the Activin A is added at a higher concentration than the BMP4. In some embodiments the BMP4 is added at a concentration of 10 ng/ml and the Activin A is added at a concentration of 12 ng/ml. In some embodiments the BMP component and the Activin component are added at day 1 of the process. In some embodiments, the concentration of the BMP component and/or the Activin component are determined by measuring for the presence or quantity of CD235a. In some embodiments the concentration of the BMP component and/or the Activin component are determined by measuring for the presence or quantity of RALDH2. In some embodiment the concentration of the BMP component and/or the Activin component are determined by measuring for the level of CD235a and comparing this to the level of RALDH2. In some embodiments the concentration of the Activin component is chosen on the basis of the concentration which preferentially results in more CD235a expressing mesoderm cells as compared with RALDH2 expressing cells, and the BMP component is added at a lower concentration than the concentration of the Activin component. In some embodiments the concentration of the BMP component is chosen on the basis of the concentration which preferentially results in more CD235a as compared with RALDH2, and the Activin component Is added at a higher concentration than the concentration of the BMP component.

Characterization of Cardiomyocytes Generated from Different Mesoderm

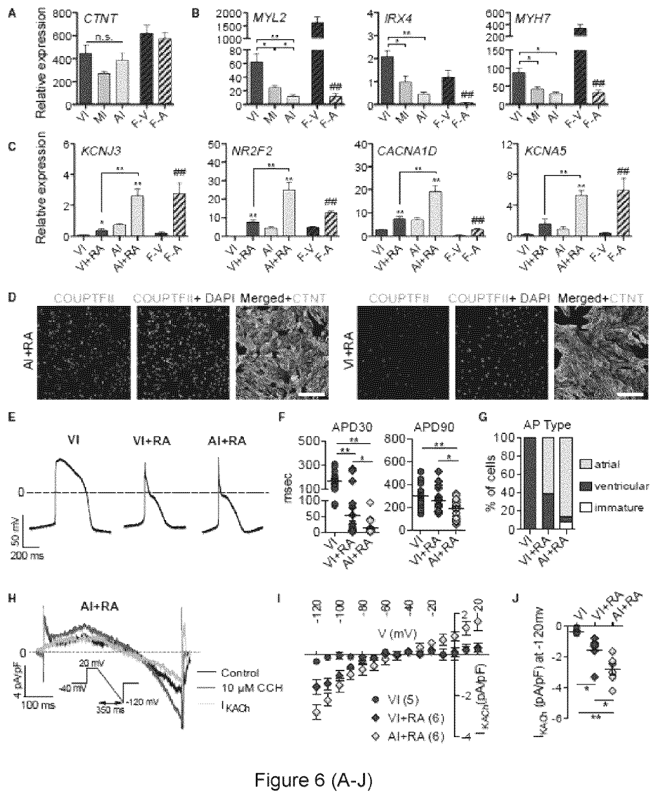

To further investigate the cardiogenic potential of the different mesoderm populations, we isolated day 20 NKX2-5+SIRPalpha+CD90− cardiomyocytes generated from EBs induced with our original cytokine combination (10B/6A; mixed induced MI) or with combinations optimized for ventricular (5B/12A; ventricular induced VI) or atrial (3B2A; atrial induced AI) fates. As expected, the expression levels of CTNT were similar in the sorted populations (FIG. 6A). Cardiomyocytes generated from the VI EBs expressed significantly higher levels of genes associated with ventricular myocytes including MYL2, IRX4 and MYH7, than cardiomyocytes derive from MI or AI EBs (FIG. 6B). Populations derived from VI Ebs had the highest frequency of MCL2V+ cardiomyocytes. (80% ±2% from VI EBs, 56% ±4% from MI EBs, and 25% ±5% from AI EBs), suggesting that the improved ventricular expression profile is due, in part, to the enriched frequency of ventricular-like cardiomyocytes (FIG. 13A). Immunostaining analyses confirmed the differences in MLC2V content of the cardiomyocyte populations (FIG. 13B).

Cardiomyocytes generated from RA-treated VI and AI EBs (VI+RA and AI+RA, respectively) showed elevated levels of expression of all the atrial genes analyzed compared to those isolated from the non-treated EBs (FIGS. 8C and 13C). The levels of expression of KCNA5, KCNJ3, NR2F2 and CACNA1D in the cells from the AI+RA were as high as or higher than those in the fetal atrial tissue (FIG. 6C). Notably, their expression levels were also significantly higher than those detected in the myocytes generated from the VI+RA EBs. In contrast, other atrial genes, such as GJA5, NPPA, and MYL7, were expressed at comparable levels in the two RA-treated cardiomyocyte populations but at significantly lower levels than those detected in the fetal atrial tissue (FIG. 14C). The levels of the pacemaker gene TBX3 were comparable in the two RA-treated groups, indicating that the observed differences in KCNA5, KCNJ3, CACNA1D, and NR2F2 expression were not due to contaminating pacemaker cells in the atrial population (FIG. 13D).

Given that CD235a+ mesoderm expresses CYP26A1 that can degrade RA, it Is possible that the differences In expression of the atrial genes are due to differences in the final concentration of active ligand that reaches the nuclear receptors. To test this, we varied the concentration of RA used for atrial specification and analyzed isolated NKX2-5+SIRPa+ cells (day 20) generated from each EB induction condition (FIG. 13E). Increasing the concentration of RA from 0.5 to 1-2 mM did increase the expression level of KCNA5 in the cardiomyocytes from the VI EBs to levels comparable to the cells from the AI EBs (FIG. 13F). These concentrations of RA were also sufficient to completely suppress the expression of the ventricular genes MYL2 and IRX4 in the VI population (FIG. 13G). In contrast, addition of RA at concentrations of up to 2 mM failed to increase the expression of KCNJ3, CACNA1D, and NR2F2 in VI cardiomyocytes to the levels observed in AI cells (FIG. 14H). Comparable expression levels of these genes were only detected in cardiomyocytes generated from EBs treated with 4 mM RA, a concentration that resulted in a dramatic reduction in the frequency of NKX2-5+SIRPa+ cells in the day 20 populations (FIG. 14E). These data further demonstrate that the VI and AI mesoderm populations do not have the same potential. Additionally, they highlight the importance of using appropriate early-stage induction strategies for the efficient specification of ventricular and atrial cardiomyocytes. To assess whether the above populations differed functionally. we tested the electrophysiological characteristics of the NKX2-5+SIRPa+CD90-cardiomyocytes derived from VI and AI t RA EBs using patch-clamp experiments.

TABLE 1
(Related to FIG. 6) Elerophysiological characteristics
of the cardio-myocytes derived from VI and AI EBs

| VI | | | |
|---|---|---|---|
| spontaneous | stimulated | VI + RA | AI + RA |
| (n = 15) | (n = 3) | (n = 18) | (n = 20) |

AP characteristics

| | | | | |
|---|---|---|---|---|
| DMP (mV) | −57.0 ± 2.3 | −70.0 ± 4.2* | −54.3 ± 1.9 | −54.6 ± 1.7 |
| APA (mV) | 91.5 ± 4.1 | 81.7 ± 4.1 | 80.0 ± 5.2 | 84.5 ± 5.2 |
| dv/dtₘₐₓ (V/s) | 55.8 ± 6.4 | 48.9 ± 3.5 | 67.1 ± 7.6 | 67.7 ± 12.2 |
| APD30 (ms) | 172 ± 18 | 133 ± 23 | 55 ± 20** | 13.0 ± 4.8** [symbol] |
| APD90 (ms) | 320 ± 32 | 227 ± 30 | 258 ± 25 | 189 ± 18** [symbol] |
| CL (s) | 3.7 ± 0.7 | n.a. | 1.1 ± 0.1** | 0.75 ± 0.1** [symbol] |

Classification into AP types

| | | | | | |
|---|---|---|---|---|---|
| ventricular (%) | 100 ± 0 (18 cells) | 38 ± 5 | (6 cells)** | 6 ± 6 | (2 cells)** [symbol] |
| atrial (%) | 0 ± 0 | 62 ± 5 | (12 cells)** | 86 ± 9 | (17 cells)** |

| pacemaker (%) | 0 ± 0 | 0 ± 0 | 0 ± 0 | |
|---|---|---|---|---|
| immature (%) | 0 ± 0 | 0 ± 0 | 8 ± 8 | (1 cell) |

[symbol]

indicates data missing or illegible when filed

APA, action potential amplitude: APD30/90, action potential duration at 30%/90% of repolarization; CL, cycle length; DMP, diastolic membrane potential; dv/dtmax, maximum action potential upstroke velocity; t-test *P<0.05, **P<0.01 vs VI spontaneous and #P<0.05, ##P<0.01 vs VI+RA. % of AP type ±SEM was quantified from cell batches patched of n=5 (VI, VI+RA) and n=6 (AI+RA) independent differentiation. Details on the parameters used for the classification into AP types are specified in the methods section.

As flow cytometric analysis for MLC2V had already demonstrated a low efficiency of specification of ventricular cardiomyocytes from AI EBs in the absence of RA, these cardiomyocytes were not further analyzed in the patch-clamp experiments. VI EB-derived cardiomyocytes (in the absence of RA) showed typical ventricular action potentials (APs) with fast upstroke velocities (>10 V/s) and long AP durations (APD30>50 ms) (FIGS. 6E and 6F). Importantly, 100% of the analyzed cells showed this ventricular phenotype (FIG. 6G). Cardiomyocytes that were specified from VI or AI EBs in the presence of RA displayed significantly faster beating rates and shorter APD30s compared to VI EB-derived cardiomyocytes, Indicative of an atrial AP phenotype (FIGS. 6E and 6F). How-ever, the APD30 and APD90 of VI+RA EB-derived cardiomyocytes were significantly longer than found in AI+RA EB-derived cardiomyocytes (APD30. 55±20 ms versus 13.0±4.8 ms; APD90, 258±25 ms versus 189±18 ms). Classification of the observed AP types revealed striking differences in the proportion of atrial and ventricular-like APs re-corded in the cells from the two groups (FIG. 6G). Only 62%±5% of the cells analyzed from the VI+RA EBs showed an atrial pattern, with the remaining 38% ±5% displaying a ventricular phenotype (APD30/90>0.3). In contrast, the majority (86%±9%) of the cells in the AI+RA EBs showed an atrial pattern with only 6% 6% displaying a ventricular AP. One cell of 20 recorded from the AI+RA EBs had a slow upstroke velocity (<10 V/s) and slow beating rate (50 bpm), indicative of an Immature cardiomyocyte. To further characterize the atrial cells generated from the two EB populations, we next measured acetylcholine-activated potassium current densities (IKACh), focusing only on cells that dis-played an atrial AP phenotype (upstroke velocity >10 V/s, APD30/90<0.3). As expected, control VI EB-derived ventricular cells (-RA) displayed significantly smaller IKACh current density than the atrial cells generated from both populations (FIGS. 6H-6J). Comparison of the two atrial cardiomyocyte populations revealed interesting differences, as those derived from AI+RA EBs showed significantly higher IKACh current densities than those derived from VI+RA EBs (2.8±0.4 pA/pF versus 1.6±0.4 pA/pF). Taken together with the above observations, these findings indicate that the efficiency of generating atrial cells and the quality of these cells is dependent on generating the appropriate mesoderm population.

Generation of Atrial and Ventricular Cardiomyocytes from Other hPSC Lines.

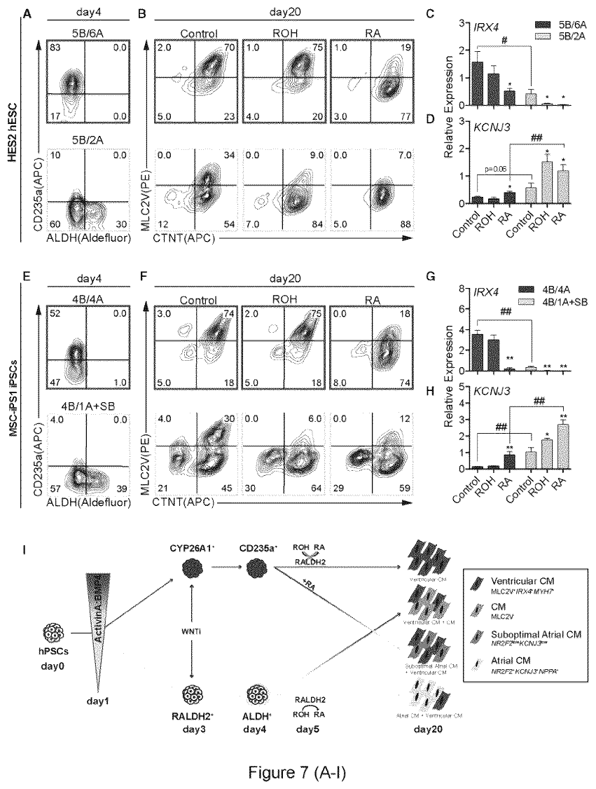

To determine if the approach for optimizing atrial and ventricular differentiation based on ALDH activity and CD235a expression is broadly applicable, we next used it to generate these cardiomyocyte populations from the HES2 human embryonic stem cell and the MSC-iPS1 induced pluripotent stem cell lines. Titration studies identified 5B/2A and 5B/6A as optimal for atrial and ventricular inductions, respectively, for HES2 cells and 4B/4A as optimal for ventricular induction for MSC-iPSC1 cells (FIGS. 7A, 7B, 7E, and 7F; Mendeley http://dx.doi.org/10.17632/7z7d5v2c3w.1). Optimization of atrial induction from the MSC-iPSC1 cells was more challenging, as all cytokine combinations promoted the development of a substantial CD235a+ population. One interpretation of these patterns is that the MSC-iPS1 cells have a high level of endogenous Nodal/Activin A signaling, resulting in the development of some CD235a+ cells under all conditions. To test this, we added the Nodal/Activin A/transforming growth factor beta (TGF-beta) inhibitor SB-431542 (SB) from days 3 to 5 to cells induced with 4B/1A. SB addition did lead to a reduction in CD235a+ cells and an increase in the size of the ALDH+ population without affecting the CTNT+MLC2V− cardiogenic potential of the day 4 mesoderm (FIGS. 7E, 7F, and 14A), supporting the interpretation that the MSC-iPS1 cells have higher levels of endogenous Nodal/Activin A signaling than the other lines.

EBs optimized for CD235a+ mesoderm development from both lines generated day 20 populations that contained high proportions of MLC2V+CTNT+ cardiomyocytes that expressed IRX4 (FIGS. 7B, 7C, 7F, and 7G). Neither CD235a+ mesoderm population responded to ROH. As expected, both responded to RA, and they generated cardiomyocyte populations that showed reduced MLC2V content, a downregulation of MYL2 and IRX4 expression, and an upregulation of KCNJ3 and NR2F2 compared to the untreated controls (FIGS. 7B-7D, 7F-7H, and 14B-14G). The EBs optimized for ALDH+ mesoderm development responded to both ROH and RA, and they generated cardiomyocyte populations that displayed expression profiles indicative of the atrial linage (FIGS. 7B-7D, 7F-7H, and 14B-14G). Taken together, these findings demonstrate that ALDH+ and CD235a+ mesoderm populations generated from the different hPSC lines display atrial and ventricular potential, respectively, similar to the populations generated from the HES3-NKX2-5eGFP/w line. We used the hPSC differentiation system to model the earliest stages of human cardiac development, with the goal of mapping the emergence and segregation of the atrial and ventricular cardiomyocyte lineages. The findings from this work support a scheme of human cardiac development in which atrial and ventricular cardiomyocytes derive from distinct mesoderm populations that are specified by different levels of Activin A and BMP4 signaling and can be identified based on ALDH activity (RALDH2) or CD235a/CYP26A1 expression, respectively (FIG. 71). We propose that atrial cardiogenesis is induced via autocrine RA signaling within a subpopulation of RALDH2⁺ mesoderm, whereas inhibition of the pathway in CD235a⁺ mesoderm through expression of CYP26A1 is required for ventricular cardiomyocyte development. Although the RALDH2⁺ and CD235a⁺ populations can give rise to both types of cardiomyocytes, the efficient generation of atrial and ventricular cells is dependent upon induction of the appropriate mesoderm. Collectively, these new insights provide a framework for accessing the earliest stages of human cardiac development and a platform for designing optimal protocols for the efficient generation of specific cardiomyocyte subtypes.

Our observation that atrial specification is mediated by RA signaling during the mesoderm stage of development is consistent with previous reports on atrial differentiation from hPSCs (Devalla et al., 2015; Zhang et al., 2011) as well as with the time-restricted effect of RA on cardiogenesis described in the early embryo (Moss at al., 1998; Xavier-Neto at al., 2000). In the embryo, this stage correlates with the emergence of a population of RA-responsive and RALDH2-expressing cells in the lateral plate mesoderm that is thought to contribute to the posterior region of the heart tube and ultimately gives rise to atrial cardiomyocytes (Hochgreb at al., 2003; Moss at al., 1998). The highly overlapping patterns of RA responsiveness and RALDH2 expression suggest that this mesoderm can both synthesize and respond to RA The concept that a subpopulation of cardiac mesoderm in vivo can synthesize RA is supported by the study of Lescroart et al. (2014), which showed that the migrating Mesp1⁺ mesoderm (E7.25) that contributes to atria development ex-presses significantly higher levels of ALDH1A2 (RALDH2) than the early migrating ventricular progenitors (E6.25-6.75). The findings from our cell-sorting experiments dearly demonstrate that 3B/2A-induced mesoderm with atrial potential does express RALDH2 and is able to respond to ROH, providing compelling evidence that human atrial specification is mediated through autocrine RA signaling.

The finding that CD235a⁺CYP26A1⁺ALDH⁻ mesoderm efficiently generates ventricular cardiomyocytes but is unable to respond to ROH to generate atrial cells provides strong evidence that these cardiomyocyte subtypes derive from different mesoderm populations. The differential expression of CYP26A1 and RALDH2 in the CD235a⁺ and ALDH⁺ mesoderm indicates that these hPSC-derived progenitors have established the balance between RA synthesis and degradation similar to the RA-signaling boundaries found along the anterior-posterior axis of the cardiovascular progenitor field in developing embryos (Cunningham and Duester, 2015; Rydeen and Waxman. 2014). Currently, it is not known if the CD235a mesoderm generates left or right ventricular cardiomyocytes or a mixture of both. Until we are able to achieve better resolution of these populations in vitro, it is difficult to incorporate our findings into the first and second heart field model that proposes that different progenitors contribute to the left ventricle and the right ventricle outflow tract (Buckingham at al., 2005; Meilhac et al., 2004; Spaeter at al.. 2013). Our findings are, however, in line with those of Bardot at al. (2017), who used a lineage-tracing strategy to show that expression of FOXA2 in the mouse marks progenitors that give rise to left and right ventricular, but not atrial, cardiomyocytes.

The ability to monitor ventricular and atrial progenitor development quantitatively through CD235a expression and ALDH activity enabled us to investigate the pathways that regulate the specification of these two populations and to demonstrate that gradients of BMP4 and Activin A signaling play a pivotal role in these early decisions. Our analyses of different hPSC lines revealed that specification of the ventricular lineage is dependent on a higher ratio of Activin A to BMP4 signaling than is required for the generation of the atrial lineage. These differences may reflect the different signaling environments that these progenitors are exposed to in the early embryo. Evidence in support of this is provided by the study of Lescroart et al. (2014), which showed that transcripts for Nodal and its downstream target genes PITX2, LEFT1, FGF8, GSC, and MIX)(Lee at al., 2011) are enriched in the early migrating left ventricular progenitors compared to the later developing atrial progenitors.

The observation that optimal ventricular and atrial development is dependent on the efficient specification of the appropriate mesoderm underscores the importance of understanding the earliest stages of development in the hPSC differentiation cultures. Our findings show that both the efficiency of lineage development and, in the case of atrial cardiomyocytes, the quality of the cells generated are influenced by the early induction steps. The precise control of lineage development in the differentiation cultures has important implications for translating the potential of hPSCs to therapeutic applications for cardiovascular disease. For instance, the highly enriched ventricular cardiomyocytes, devoid of contaminating pacemaker and atrial cells, would be an ideal candidate population for developing cell-based therapies aimed at remuscularization of the ventricular wall in patients suffering from a myocardial infarction. Elimination of the non-ventricular cells may reduce the arrhythmias observed in animal models following transplantation of mixed populations of hPSC-derived cardiomyocytes (Chong et al., 2014; Shiba at al., 2018). Access to enriched populations of cardiomyocyte subtypes is also important for modeling diseases that affect specific regions of the heart, such as atrial fibrillation, hypertrophic cardiomyopathy, and other chamber-specific congenital heart defects. The ability to generate different cardiac populations will not only provide the appropriate target cells for such studies but will also enable analyses of potential off-target effects of therapeutic strategies on the other cardiomyocyte subtypes. These comprehensive analyses will provide insights into human cardiovascular disease that are not possible with the use of poorly characterized, mixed populations.

### Methods Details

### Directed Differentiation of Human ESC/iPSC Lines

For cardiac differentiation, we used a modified version of our embryoid body (EB)-based protocol (Kattman et al., 2011). hPSC populations at 80%-90% confluence were dissociated into single cells (TrypLE, ThermoFisher) and re-aggregated to form EBs in StemPro-34 media (ThermoFisher) containing penicillin/streptomycin (1%, ThermoFisher), L-glutamine (2 mM, ThermoFisher), trans-ferrin (150 pg/ml, ROCHE), ascorbic acid (50 μg/ml, Sigma), and monothioglycerol (50 μg/ml, Sigma), ROCK inhibitor Y-27632 (10 μM, TOCRIS) and rhBMP4 (1 ng/ml, R&D) for 18 h on an orbital shaker. At day 1, the EBs were transferred to mesoderm induction media consisting of StemPro-34 with above supplements (-ROCK inhibitor Y-27632) and rhBMP4, rhActivinA (R&D) and rhbFGF (5 ng/ml, R&D) at the indicated concentrations. At day 3, the EBs were harvested, washed with IMDM and transferred to cardiac mesoderm specification media consisting of StemPro-34, the Wnt inhibitor IWP2 (1 μM, TOCRIS) and rhVEGF (10 ng/mL, R&D). At day 5, the EBs were transferred to StemPro-34 with rhVEGF (5 ng/ml) for another 7 days and then to StemPro-34 media without additional cytokines for further 8 days. At day 20, HES3-NKX2-5gfp/w-derived cardiomyocytes were analyzed and isolated based on the expression of NKX2-5:GFP and SIRPa and alack of CD90. Cardiomyocytes generated from non-transgenic hPSC lines were analyzed and isolated as SIRPa+CD90− populations. Media was changed every 3 days. Cultures were incubated in a low oxygen environment (5% CO₂, 5% O₂, 90% N₂) for first 12 days and a normoxic environment (5% CO₂) for the following 8 days in total of 20 days. The EBs were cultured in ultra-low attachment 6-well dishes (Corning) throughout the differentiation for maintaining suspension cultures.

### Optimization of Atrial and Ventricular Inductive Conditions

For determining the optimal atrial inductive conditions, the selection of Activin A and BMP4 concentrations was based on identification of a mesoderm population with the highest proportion of ALDH+CD235a− cells at day 4 that showed the greatest potential to generate CTNT+MLC2V− cardiomyocytes at day 20. Following optimization, either ATRA (0.5 μM, Sigma) or retinol (2 μM, Sigma) was included in the cardiac mesoderm specification media from days 3-5 for the generation of atrial cardiomyocytes.

For determining the optimal ventricular inductive conditions, the selection of Activin A and BMP4 concentrations was based on identification of a mesoderm population that contained a high proportion of CD235a+ cells, no ALDH+ cells and generated a high pro-portion of CTNT+MLC2V+ at day 20.

### Flow Cytometry and Cell Sorting

Day 2-8 EBs were dissociated with TrypLE for 2-4 min at room temperature (RT). Day 20 EBs were dissociated by incubation in Collagenase type 2 (0.5 ng/ml, Worthington) in HANKs buffer overnight at RT followed by TrypLE treatment as described above. The following antibodies were used for staining: anti-PDGFRa-PE (R&D Systems, 3:50), anti-CD235a-APC (BD PharMingen,1:100), antiSIRPa-PeCy7 (Biolegend,1:1000), anti-CD90-APC (BD PharMingen, 1:1000), anticardiac isoform of CTNT (ThermoFisher Scientific, 1:2000), or anti-myosin light chain 2 (Abcam,1:1000). For unconjugated primary antibodies, the following secondary antibodies were used for detection: goat anti-mouse IgG-APC (BD Pharmigen, 1:250), or donkey anti-rabbit IgG-PE (Jackson ImmunoResearch, 1:250). Detailed antibody information is described in Table 2.

TABLE 2
Experimental Resources.

| | | |
|---|---|---|
| REAGENT or RESOURCE | SOURCE | IDENTIFIER |

Antibodies

| | | |
|---|---|---|
| Mouse monoclonal to PDGFR [symbol] (clone [symbol] R1), PE conjugated | BD PharMingen | Cat. # 556002; RRID: AB_396286 |
| Mouse monoclonal to CD235a (clone HIR2), APC conjugated | BD PharMingen | Cat. # 551336; RRID: AB_398499 |
| Mouse monoclonal to SIRPα (clone SE5A5), PeCy7 | Biolegend | Cat. # 323807; RRID: AB_1236443 |
| conjugated | | |
| Mouse monoclonal to CD90 (clone 5E10), APC conjugated | BD PharMingen | Cat. # 553869; RRID: AB_398677 |
| Mouse monoclonal to CTNT (clone 13-11) | ThermoFisher | Cat. # MA5-12960; RRID: AB_11000742 |
| Rabbit polyclonal to MLC2V | Abcam | Cat. # 79935; RRID: AB_1952220 |
| Goat anti-mouse IgG (H + L), APC conjugated | BD PharMingen | Cat. # 550826; RRID: AB_398465 |
| Donkey anti-rabbit IgG (H + L), PE conjugated | Jackson ImmunoResearch | Cat. # 711-116-152; RRID: AB_2340599 |
| Mouse monoclonal to COUP-TFII (clone H7147) | R&D | Cat. # PP-H7147-00; RRID: AB_2155627 |
| Rabbit monoclonal to CTNT | Genway Biotech | Cat. # GWB-25E5E5 |
| Donkey anti-rabbit IgG (H + L), AlexaFluor555 conjugated | ThermoFisher | Cat. # A31572; RRID: AB_1 [symbol] 2543 |
| Donkey anti-mouse IgG (H + L). AlexaFluor647 conjugated | ThermoFisher | Cat. # A31571; RRID: AB_1 [symbol] 2542 |

Biological Samples

| | | |
|---|---|---|
| Human fetal heart tissues | Provided by R. Hamilton | N/A |
| | (SickKids Hospital, Canada) | |

Chemicals, Peptides, and Recombinent Proteins

| | | |
|---|---|---|
| Penicillin/streptomycin | ThermoFisher | Cat. # 15070063 |
| L-glutamine | ThermoFisher | Cat. # 25030081 |
| non-essential amino acids | ThermoFisher | Cat. # 11140-050 |
| Transferrin | ROCHE | Cat. # 10652202 |
| Ascorbic acid | Sigma | Cat. # A-45440 |
| Monothioglycerol | Sigma | Cat. # M- [symbol] 145 |
| β-Mercaptoethanol | ThermoFisher | Cat. # 21985-023 |
| ROCK inhibitor Y-27632 | Tocris | Cat. # 1254 |
| Recombinant human BMP4 | R&D | Cat. # 314-BP |
| Recombinant human ActivinA | R&D | Cat. # 338-AC |
| Recombinant human bFGF | R&D | Cat. # 223-FB |
| IWP2 (Wnt inhibitor) | Tocris | Cat. # 3533 |
| Rocombinant human VEGF | R&D | cat. # 293-VE |
| All trans RA | Sigma | cat. # R2625 |
| Retinol | Sigma | Cat. # R7632 |
| SB-431542 (TGFβ inhibitor) | Sigma | Cat. # S4317-5MG |
| Collagenase type 2 | Worthington | Cat. # 4176 |
| AM580 (RARα agonist) | Tocris | Cat. # 0760 |
| AC55649 (RARβ agonist) | Tocris | Cat. # 2438 |
| CD437 (RARγ agonist) | Tocris | Cat. # 1549 |
| Fetal calf serum (FCS) | Wisent | Cat. # 088-150 |
| Bovine serum albumin (BSA) | Sigma | Cat. # A2153 |
| Matrigel, growth factor reduced | Corning | Cat. # 356230 |
| Glycine | Sigma | Cat. # G2289 |
| SlowFade gold antifade with DAPI | ThermoFisher | Cat. # S36939 |

Critical Commercial Assays

| | | |
|---|---|---|
| Aldefluor assay kit | STEMCELL Technologies | Cat. # 1700 |
| RNAqueous-micro kit with RNase-free DNase treatment | Ambion | Cat. # AM1931 |
| TRIzol | ThermoFisher | Cat. # 15596026 |
| Superscript III Reverse Transcriptase kit | ThermoFisher | Cat. #18080044 |
| QuantiFast SYBR Green PCR kit | QIAGEN | Cat. # 204145 |

Deposited Data

| | | |
|---|---|---|
| Optimization data of HES2 hESC and MSC-iPS1 hiPSC lines | This paper; Mendeley Data | [symbol] |

Experimental Models: Cell Lines

| | | |
|---|---|---|
| Human ESC: HES3 line | Gift from Drs. E. Stanley and | N/A |
| | A. Elefanty, Monash University, | |
| | AU (EIliott et al.. 2011) | |
| Human ESC: HES2 line | WiCell | Cat. # ES02 |
| Human iPSC; MSC-iPSC1 line | Gift from Dr. G. Daley, Harvard | N/A |
| | Medical School, US (Park et al.. | |
| | 2008) | |

Oligonucleotides

| | | |
|---|---|---|
| See Table S2 for PCR primer sequences | This paper | Table S2 |

Software and Algorithms

| | | |
|---|---|---|
| pCLAMP | Molecular Devices | [symbol] |
| FlowJo | Tree Star | [symbol] |
| FV10-ASW | Olympus | [symbol] |
| MultiExperiment Viewer | MeV | [symbol] |
| Graphpad Prism 6 | GraphPad Software | [symbol] |

Other

| | | |
|---|---|---|
| StemPro-34 media | ThermoFisher | Cat. # 10640019 |
| DMEM/F12 | Cellgro | Cat. # 10-092-CV |
| KnockOut serum replacement | ThermoFisher | Cat. # 10828028 |
| TrypLE | ThermoFisher | Cat. # 12605010 |

[symbol]

indicates data missing or illegible when filed

For cell-surface marker analyses. cells were stained for 30 min at 4 degrees C. in FACS buffer consisting of PBS with 5% fetal calf serum (FCS) (Wisent) and 0.02% sodium azide. For intracellular staining, cells were fixed for 15 min at 4 degrees C. with 4% PFA in PBS followed by permeabilization using 90% methanol for 20 min at 4 degrees C. Cells were washed with PBS containing 0.5% BSA (Sigma) and stained with unconjugated primary antibodies in FACS buffer overnight at 4 degrees C. Stained cells were washed with PBS with 0.5% BSA and stained with secondary antibodies in FACS buffer for 1 h at 4 degrees C.

Stained cells ware analyzed using the LSR II Flow cytometer (BD). For cell sorting, stained cells were kept in IMDM with 0.5% FCS and sorted using Influx (BD), FACSArialI (BD), MoFlo-XDP (BD) and FACSAria Fusion (BD) at the Sickids/UHN flow cytometry facility. Data were analyzed using FlowJo software (Tree Star).

### Aldefluor Assay

The Aldefluor™ assay (STEMCELL Technologies) was performed according to the instruction provided by the manufacturer. Briefly, day2-6 EBs were dissociated as described above. Cells were stained at a concentration of 2×106 cells/ml in the aldefluor assay buffer containing 0.1% BSA and BAAA substrate (0.12 mg/ml) for 60 min at 37 degrees C. The aldehyde dehydrogenase inhibitor DEAB (0.75 nM) was added to the negative control sample. Cells were washed with cold media consisting of IMDM with 5% FCS and 10% aldefluor assay buffer. Cells were then stained with antibodies to cell surface markers at the concentrations indicated above in cold wash media for additional 20 min at 4 degrees C. Stained cells were analyzed as described above. During analyses, the cells were kept in cold wash media. For cell sorting, FCS was replaced with KnockOut™ serum replacement (ThermoFisher) to avoid any impact of serum-contained cy-tokines on the cell differentiations. Cells were maintained in StemPro-34 containing 10% aldefluor assay buffer throughout the sorting procedure. The sorted cells were collected and re-aggregated in StemPro-34 containing ROCK inhibitor (10 μM), IWP2 (0.5 μM) and rhVEGF (5 ng/ml).

### Immunohistochemistry

Day20 EBs were dissociated as described above and the cells plated onto 12 mm cover glasses (VWR) pre-coated with matrigel (25% v/v, BD). Cells were cultured for 3-5 days to enable the formation of adherent cell monolayers. Cells were fixed with

4% PFA in PBS for 10 min at room temperature and permeabilized with PBS containing 0.3% TritonX, 200 mM Glycine (Sigma) for 20 min at RT. Cells were blocked with PBS containing 10% FCS, 0.1% TritonX, and 2% BSA. The following antibodies were used for staining: mouse anti-cardiac isoform of CTNT (ThermoFisher Scientific, 1:200), rabbit anti-human/rodent myosin light chain 2 (Abcam, 1:200), mouse anti-human COUPTF-II (R&D, 1:1000), or rabbit anti-human CTNT (Genway Biotech Inc., 1:1000). For detecting unconjugated primary antibodies, the following secondary antibodies were used: donkey anti-mouse IgG-A647 (ThermoFisher, 1:1000), or donkey anti-rabbit IgG-A555 (ThermoFisher, 1:1000). Detailed antibody information is described in the Key Resources Table. Cells were stained with primary antibodies in staining buffer consisting of PBS with 0.1% TritonX, and 0.1% BSA overnight at 4 degrees C. The stained cells were washed with staining buffer for 15 min at RT on an orbital shaker. The cells were then stained with secondary antibodies in staining buffer for 1 h at RT followed by a wash step as described above. The samples were mounted using SlowFade Gold Antifade reagent with DAPI (ThermoFisher). Stained cells were analyzed using an Olympus FluoView 1000 Laser Scanning Confocal Microscope. FV10-ASW softwarewas used forimage acquisition.

### Quantitative Real-Time PCR

Total RNA from hPSC-derived populations was isolated using RNAqueous-micro Kit including RNase-free DNase treatment (Ambion). RNA from dissected ventricular and atrial tissue of human fetal hearts was isolated using the TRlzol method (ThermoFisher) and treated with DNase (Ambion). Between 100 ng and 1 mg of isolated RNA was reverse transcribed into cDNA using oligo (dT) primers and random hexamers and Superscript III Reverse Transcriptase (ThermoFisher). QRT-PCR was performed on an EP Real-Plex MasterCycler (Eppendorf) using QuantiFast SYBR Green PCR kit (QIAGEN). All experiments were prepared in duplicates and included a 10-fold dilution series of sonicated human genomic DNA standards ranging from 25 ng/ml to 2.5 μg/ml for evaluating the efficiency of PCR reaction and the copy number of each gene relative to the house keeping gene TBP. Heatmaps of gene expression data were generated using the MultiExperiment Viewer (MeV) open source software.

### Patch Clamp

For electrophysiological characterization using patch clamp, EBs were dissociated and NKX2-5+SIRPa+CD90− cardiomyocytes were isolated by FACS as described above. Isolated cells were suspended in StemPro-34 media supplemented with ROCK inhibitor (10 mM) at 1.25-5×105 cells/ml and filtered through a 70 mm filter. Drops of 40 ul of this cell suspension were applied to glass coverslips (3×5 mm) that were pre-coated with Matrigel (10% v/v) in 30 mm dishes. The cells were incubated in the 40 mL volume for 16-18 h to facilitate cell attachment. The dishes were then flooded with 2 ml of StemPro-34 media. The media was changed every 4 days. Cultures were used for patch clamp recordings between 7 to 14 days following plating. APs and membrane currents were measured using standard patch-clamp techniques in current- and voltage-clamp modes, respectively (Axopatch 2008, Molecular Devices). Voltages and currents were recorded with 5 KHz sampling rate (DigiData, Molecular Devices) and analyzed with pCLAMP software (Molecular Devices). Borosilicate glass microelectrodes were used with tip resistances of 2-5MU when filled with pipette solution. Series resistance were compensated by rv70%. APs and membrane currents were recorded at RT using the whole-cell ruptured patch method with the following bath solution (mM): NaCl 140, KCl 5.4, CaCl2 1.2, MgCl2 1, glucose 10, and HEPES 10 (pH 7.4, adjusted with NaOH). The pipette solution consisted of (mM): potassium aspartate 120, KCl 20, NaCl 5, MgATP 5 and HEPES 10 (pH 7.2, adjusted with KOH).

In quiescent cardiomyocytes APs were elicited by 1-3 ms-long depolarizing current pulses of 5-15 pA at a frequency of 1 Hz. Spon-taneous and stimulated APs were classified based on the following parameters; pacemaker-like: dv/dtmax <10 V/s, atrial-like: dv/dtmax R 10 V/s and APD30/90<0.3, ventricular-like: dv/dtmax R 10 V/s and APD30/90 R 0.3. The acetylcholine activated potassium current (IKACh) was characterized as a CCh-sensitive current (activated by CCh). Currents were measured before and after addition of carbachol (CCH, 10 mM) in response to a 350 ms voltage ramp protocol ranging from 20 mV to −120 mV from a holding potential of −40 mV (see voltage protocol inset in respective original current trace). IKACh was quantified by subtraction of the current recorded without CCh from the current recorded in the presence of CCh.

### Quantification and Statistical Analysis

All data are represented as mean t standard error of mean (SEM). Indicated sample sizes (n) represent biological replicates including independent cell culture replicates and individual tissue samples. For single cell data (beating rate quantification and patch-clamp data) samples size (n) represents the number of cells analyzed from R three independent experiments. No statistical method was used to predetermine the samples size. Due to the nature of the experiments, randomization was not performed and the investigators were not blinded. Statistical significance was determined by using Student's t test (unpaired, two-tailed) in GraphPad Prism 6 software. Results were considered to be significant at p<0.05 (*/#) and very significant at p<0.01 (**/##). All statistical parameters are reported in the respective figures and figure legends.

Although preferred embodiments of the invention have been described herein, it will be understood by those skied in the art that variations may be made thereto without departing from the spirit of the invention or the scope of the appended claims. All documents disclosed herein, including those in the following reference list, are incorporated by reference.

1-53. (canceled)

54. A method of producing a population of cardiomyocytes enriched for;
(A) ventricular cardiomyocytes, the method comprising:
(i) incubating pluripotent stem cells in mesoderm induction medium, the mesoderm induction medium comprising a BMP component and an effective amount of an activin component sufficient to generate ventricular mesoderm;
and thereafter
(ii) culturing the incubated cells in suitable medium(s) to generate a population of cardiomyocytes enriched for ventricular cardiomyocytes,
(iii) wherein optionally the pluripotent stem cells are incubated in a medium suitable for aggregate and/or embryoid body formation prior to incubating the pluripotent stem cells in the mesoderm induction medium;
(B) atrial cardiomyocytes, the method comprising:
(i) incubating pluripotent stem cells in mesoderm induction medium, the mesoderm induction medium comprising a BMP component and an effective amount of an activin component sufficient to generate atrial mesoderm;
and thereafter
(ii) adding a retinoic acid component to the cells, wherein the addition of the retinoic acid component occurs during or after the incubation in mesoderm induction medium; and
(iii) culturing the incubated cells so that a population of cardiomyocytes enriched for atrial cardiomyocytes is generated
(iv) wherein, optionally, the pluripotent stem cells are incubated in a medium suitable for aggregate and/or embryoid body formation prior to incubating the pluripotent stem cells in the mesoderm induction medium; or
(C) sinoatrial nodal pacemaker cells or epicardial cells, the method comprising:
(i) incubating pluripotent stem cells in mesoderm induction medium, the mesoderm induction medium further comprising a BMP component and an activin component in amounts sufficient to generate ALDH+/CD235− mesoderm; and thereafter
(ii) culturing the incubated cells in suitable medium(s) with one or more of WNT, FGF inhibitor and BMP to generate a population of cardiomyocytes enriched for sinoatrial nodal pacemaker cells or epicardial cells.

55. The method of claim 54, wherein in (A):
(a) the concentration of the activin component is greater than the concentration of the BMP component;
(b) the ratio of the BMP component to the activin component is about 0.3-1:1, about 0.5:1, or about 0.8:1;
(c) the concentration of the activin component is determined by measuring the level of CD235a-expressing mesoderm cells and comparing this to the level of RALDH2-expressing mesoderm cells;
(d) the concentration of the activin component is chosen by determining a concentration which preferentially results in more CD235a-expressing mesoderm cells as compared with RALDH2-expressing mesoderm cells;
(e) the activin component is added in an amount of from about 4 ng/ml to about 20 ng/ml;
(f) the concentration of the activin component is between 6-20 ng/ml;
(g) the concentration of the BMP component is between about 3 ng/ml to about 20 ng/ml;
(h) the concentration of the BMP component is 5 ng/ml or 10 ng/ml;
(j) the concentration of the activin component is 12 ng/ml;
(k) the BMP component is BMP4;
(l) the activin component is activin A;
(m) at least a portion of the population of cardiomyocytes generated is used to treat a subject in need of cardiac repair; and/or
(n) at least a portion of the population of cardiomyocytes generated is used to treat a subject in need of cardiac repair, optionally,
wherein the subject in need of cardiac repair is at risk of heart failure, suffering heart failure and/or suffering a myocardial infarction event; further optionally wherein the treatment is before, during, or after a myocardial infarction event.

56. The method of claim 54, wherein in (B) the retinoic acid component is added when the cells are RALDH2 positive and CD235a negative, and optionally:
(a) the ratio of the BMP component to the activin component is about 1.5:1 or greater, or is 3:2;
(b) the BMP component is present at a concentration of from about 3 ng/ml to about 100 ng/ml, or at a concentration of about 3 ng/ml;
(c) the activin component is present at a concentration of from about 0.01 ng/ml to about 6 ng/ml, or at a concentration of about 2 ng/ml;
(d) the retinoic acid component is trans retinoic acid or retinal;
(e) the retinoic acid component is added in a concentration of between 50 nm and 5 μM, or at a concentration of 500 nM;
(f) the BMP component is BMP4;
(g) the activin component is Activin A;
(h) the BMP component is added to the mesoderm induction medium after one day, or the activin component is added to the mesoderm induction medium after one day;
(j) the retinoic acid component is added at about day 3 to day 5 of the method;
(k) additional BMP component is not added to the mesoderm induction medium at day 3 of the method; and/or
(l) an FGF inhibitor is excluded from the mesoderm induction medium at day 3 of the method.

57. The method of claim 54 further comprising screening or evaluating the potential cardiac toxicity of a test compound or agent, comprising the steps of exposing a population of cardiomyocytes to the test compound and evaluating the viability, contractility, changes in electric potentials and/or other functionalities of the cells.

58. The method of claim 55, wherein the enriched population of ventricular cardiomyocytes comprises at least 50% of atrial cardiomyocytes, at least 60% of atrial cardiomyocytes, at least 70% of atrial cardiomyocytes, at least 80% of atrial cardiomyocytes, or at least 90% of atrial cardiomyocytes.

59. The method of claim 56, wherein the enriched the population of atrial cardiomyocytes comprises at least 50% of atrial cardiomyocytes, at least 60% of atrial cardiomyocytes, at least 70% of atrial cardiomyocytes, at least 80% of atrial cardiomyocytes, or at least 90% of atrial cardiomyocytes.

60. The method of claim 54(A), wherein the enriched population of ventricular cardiomyocytes comprises at least 50% of ventricular cardiomyocytes, at least 60% of ventricular cardiomyocytes, at least 70% of ventricular cardiomyocytes, at least 80% of ventricular cardiomyocytes, or at least 90% of ventricular cardiomyocytes, wherein the population is an isolated population; optionally, wherein the population is essentially free of pacemaker cells or devoid of pacemaker cells

61. The method of claim 55, wherein the enriched population of ventricular cardiomyocytes comprises at least 50% of ventricular cardiomyocytes, at least 60% of ventricular cardiomyocytes, at least 70% of ventricular cardiomyocytes, at least 80% of ventricular cardiomyocytes, or at least 90% of ventricular cardiomyocytes,
wherein the population is an isolated population; and
optionally, wherein the population is essentially free of pacemaker cells or devoid of pacemaker cells.

62. The method of claim 54, further comprising treating a subject in need of cardiac repair, comprising administering to the subject the population of cardiomyocytes,
optionally, wherein the subject is at risk for heart failure, is suffering heart failure and/or has experienced a myocardial infarction event, and
wherein optionally the myocardial infarction is in the ventricle of the patient.

63. The method of claim 60 further comprising treating a subject in need of cardiac repair, comprising administering to the subject the population of cardiomyocytes,
optionally, wherein the subject is at risk for heart failure, is suffering heart failure and/or has experienced a myocardial infarction event, and
wherein optionally the myocardial infarction is in the ventricle of the patient.

64. The method of claim 60, further comprising treating a subject in need of cardiac repair, comprising administering to the subject the population of cardiomyocytes,
optionally, wherein the subject is at risk for heart failure, is suffering heart failure and/or has experienced a myocardial infarction event, and
wherein optionally the myocardial infarction is in the ventricle of the patient.

65. The method of claim 61, further comprising treating a subject in need of cardiac repair, comprising administering to the subject the population of cardiomyocytes,
optionally, wherein the subject is at risk for heart failure, is suffering heart failure, and/or has experienced a myocardial infarction event, and
wherein optionally the myocardial infarction is in the ventricle of the patient.
